SEQUENCE LISTING (3 sequences)

SEQ ID NO: 1 - protein, 1675 aa, Homo sapiens
MAQILPIRFQEHLQLQNLGINPANIGFSTLTMESDKFICIREKVGEQAQVVIIDMNDPSNPIRRPISADSAIMNPASKVIALKAGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGQPQLMLTAGPSVAVPPQAPFGYGYTAPPYGQPQPGFGYSM

SEQ ID NO: 2 - protein, 248 aa, Homo sapiens
MAELDPFGAPAGAPGGPALGNGVAGAGEEDPAAAFLAQQESEIAGIENDEAFAILDGGAPGPQPHGEPPGGPDAVDGVMNGEYYQESNGPTDSYAAISQVDRLQSEPESIRKWREEQMERLEALDANSRKQEAEWKEKAIKELEEWYARQDEQLQKTKANNRVADEAFYKQPFADVIGYVTNINHPCYSLEQAAEEAFVNDIDESSPGTEWERVARLCDFNPKSSKQAKDVSRMRSVLISLKQAPLVH

SEQ ID NO: 3 - protein, 229 aa, Homo sapiens
MADDFGFFSSSESGAPEAAEEDPAAAFLAQQESEIAGIENDEGFGAPAGSHAAPAQPGPTSGAGSEDMGTTVNGDVFQEANGPADGYAAIAQADRLTQEPESIRKWREEQRKRLQELDAASKVTEQEWREKAKKDLEEWNQRQSEQVEKNKINNRIADKAFYQQPDADIIGYVASEEAFVKESKEETPGTEWEKVAQLCDFNPKSSKQCKDVSRLRSVLMSLKQTPLSR

(19) United States
(12) Patent Application Publication
(10) Pub. No.: US 2024/0398978 A1
(43) Pub. Date: Dec. 5, 2024

(54) Compositions and Methods for Treating Dopamine Disorders

(71) Applicant: METAQOR LLC, Boston, MA (US)

(72) Inventors: Franco Vitaliano, Boston, MA (US); Gordana Vitaliano, Boston, MA (US)

(73) Assignee: METAQOR LLC, Boston, MA (US)

(21) Appl. No.: 18/811,560

(22) Filed: Aug. 21, 2024

Related U.S. Application Data
(63) Continuation of application No. 17/246,018, filed on Apr. 30, 2021.
(60) Provisional application No. 63/018,324, filed on Apr. 30, 2020.

(51) Int. Cl. A61K 47/69; A61K 9/00; A61K 45/06; A61K 47/60; A61P 25/30

(52) U.S. Cl. CPC A61K 47/6929; A61K 9/0019; A61K 9/0043; A61K 45/06; A61K 47/60; A61P 25/30

(57) ABSTRACT

Compositions comprising clathrin nanoparticles and methods for treating dopamine-associated addictions, disorders, and neurodegenerative diseases using the same.

### CROSS-REFERENCE TO RELATED APPLICATIONS

This application is a continuation of U.S. patent application Ser. No. 17/246,018, filed on Apr. 30, 2021 and claims priority under 35 USC § 119(e) to U.S. Patent Application Ser. No. 63/018,324, filed on Apr. 30, 2020. The entire contents of the foregoing are hereby incorporated by reference.

### SEQUENCE LISTING

This application contains a Sequence Listing that has been submitted electronically in XML format and is hereby incorporated by reference in its entirety. The name of the text file is 222338-010203CON_SL.xml and the size of the text file is 5,742 bytes. The text file was created on Aug. 21, 2024.

### BACKGROUND

Methamphetamine use disorder (MUD) represents a major chronic health problem in the US and abroad with significant neuropsychiatric, cognitive, and socioeconomic consequences. The estimated economic cost for MUD in 2019 dollars is $31 billion (Rand Co.). In 2017, 964,000 people 12 and older had MUD in the US (NIDA). About $1.3 billion per year is spent on inpatient MUD rehab, but there is only a 14% success rate for recovery after 1 year. Decreasing inpatient stay and improving treatment efficacy are #1 priorities for MUD. Multiple medications (>20) have been tested in MUD clinical trials but failed to show efficacy. There are no FDA approved treatments for this chronic disabling disorder. Therefore there is a strong need for improved therapies for treating MUD and other dopamine disorders.

### SUMMARY

Thus provided herein are methods for treating a human subject having or at risk for developing a dopamine disorder. The methods include administering to the human subject a composition comprising: (i) a DAT targeting agent; and (ii) a neurotrophin, wherein the DAT targeting agent and neurotrophin are linked to a clathrin nanoparticle.

In some embodiments, the clathrin nanoparticle comprises a clathrin cage.

In some embodiments, the clathrin nanoparticle consists of a clathrin triskelion consisting of 1 to 3 clathrin heavy chains (CHCs) and 0-3 clathrin light chains (CLCs).

In some embodiments, the clathrin nanoparticle comprises a complex consisting of 1 to 3 clathrin heavy chains (CHCs), optionally wherein one or more of the CHCs is further linked to one human clathrin light chain; In some embodiments, each of the CHCs is linked to a CLC.

In some embodiments, the CHC and CLC are human.

In some embodiments, the DAT targeting agent comprises a dopamine re-uptake inhibitor (DRI) or an anti-DAT antibody.

In some embodiments, the dopamine reuptake inhibitor (DRI) is selected from the group consisting of -Hydroxy-1-methyl-4-(4-methylphenyl)-3-piperidyl 4-methylphenyl ketone, Altropane (O-587), Amfonelic acid (WIN 25978), Amineptine, BTCP (GK-13), 3C-PEP, DBL-583, Difluoropine (O-620), GBR-12783, GBR-12935, GBR-13069, GBR-13098, GYKI-52895, Iometopane (β-CIT, RTI-55), Methylphenidate, Ethylphenidate, Modafinil, Armodafinil, RTI-229, Vanoxerine (GBR-12909), drafinil, Amantadine, Benztropine, Bupropion, Fluorenol, Ketamine, Medifoxamine, Metaphit, Rimcazole, Sertraline, St. John's Wort, Venlafaxine, Chaenomeles speciose, 3-Methoxyphencyclidine, 4-Methoxyphencyclidine, and Oroxylin A.

In some embodiments, the neurotrophin comprises BDNF, NGF, NT-3, NT-4, NT-6, GDNF, NTN, PSPN, ARTN, CNTF, or LIF.

In some embodiments, the DAT targeting agent and/or the neurotrophin is linked to the clathrin nanoparticle by conjugation.

In some embodiments, the DAT targeting agent is conjugated to the clathrin nanoparticles via a linker, e.g., via PEG.

In some embodiments, the neurotrophin is conjugated to the clathrin nanoparticles, optionally to a CHC, via PEG.

In some embodiments, the DAT targeting agent and the neurotrophin are linked to the same CHC, or to different CHCs.

In some embodiments, at least one clathrin heavy chain is linked to 1 to 5 molecules of the DAT targeting agent and/or 1 to 5 molecules of the neurotrophin.

In some embodiments, a clathrin heavy chain is linked to 1 to 5 molecules of the DAT targeting agent and 1 to 5 molecules of the neurotrophin.

In some embodiments, at least one CHC is linked to one molecule of BDNF and four molecules of a DRI, or one molecule of BDNF and three molecules of an anti-DAT antibody.

In some embodiments, the dopamine disorder is a neurodegenerative disease associated with loss of dopaminergic neurons. In some embodiments, the neurodegenerative disease associated with loss of dopaminergic neurons is Parkinson's disease, Huntington's disease, Alzheimer's disease, or ALS.

In some embodiments, the dopamine disorder is ADHD, depression, or schizophrenia.

In some embodiments, the dopamine disorder is a dopamine-driven addiction, e.g., methamphetamine use disorder, cocaine addiction, opioid addiction, sex addiction, food addiction, or gambling addiction.

In some embodiments, the composition is delivered intranasally or intravenously.

In one aspect, provided herein are compositions comprising a dopamine active transporter (DAT) targeting agent linked to a clathrin nanoparticle. In some embodiments, the clathrin nanoparticle is further linked to a neurotrophin. In some embodiments, the clathrin nanoparticle comprises a clathrin cage. In some embodiments, the clathrin nanoparticle consists of a clathrin triskelion. In some embodiments, the clathrin nanoparticle comprises a complex consisting of 1 to 3 clathrin heavy chains. In some embodiments, the DAT targeting agent comprises a dopamine re-uptake inhibitor or an anti-DAT antibody. In some embodiments, the dopamine reuptake inhibitor is Vanoxerine. In some embodiments, the neurotrophin comprises BDNF, NGF, NT-3, NT-4, NT-6, GDNF, NTN, PSPN, ARTN, CNTF, or LIF. In some embodiments, the DAT targeting agent is linked to the clathrin nanoparticles by conjugation. In some embodiments, the DAT targeting agent is conjugated to the clathrin nanoparticles via PEG. In some embodiments, the neurotrophin is linked to the clathrin nanoparticles by conjugation. In some embodiments, the neurotrophin is conjugated to the clathrin nanoparticles via PEG. In some embodiments, the DAT targeting agent and the neurotrophin are linked to the same heavy chain of the clathrin triskelion. In some embodiments, the DAT targeting agent and the neurotrophin are linked to different heavy chains of the clathrin triskelion. In some embodiments, a clathrin heavy chain of the clathrin triskelion is linked to 1 to 5 molecules of the DAT targeting agent. In some embodiments, a clathrin heavy chain of the clathrin triskelion is linked to 1 to 5 molecules of the neurotrophin. In some embodiments, a clathrin heavy chain of the clathrin triskelion is linked to 1 to 5 molecules of the DAT targeting agent and 1 to 5 molecules of the neurotrophin. In some embodiments, a clathrin heavy chain of the clathrin triskelion is linked to one molecule of BDNF and four molecules of Vanoxerine. In some embodiments, a clathrin heavy chain of the clathrin triskelion is linked to one molecule of BDNF and three molecules of an anti-DAT antibody. In some embodiments, one of the 1 to 3 clathrin heavy chains is linked to 1 to 5 molecules of the DAT targeting agent. In some embodiments, one of the 1 to 3 clathrin heavy chains is linked to 1 to 5 molecules of the neurotrophin. In some embodiments, one of the 1 to 3 clathrin heavy chains is linked to 1 to 5 molecules of the DAT targeting agent and 1 to 5 molecules of the neurotrophin.

In another aspect, provided herein are methods for treating a human subject having or at risk for developing a dopamine disorder comprising administering to the human subject a composition comprising a DAT targeting agent linked to a clathrin nanoparticle. In some embodiments of any of the methods for treating a human subject having or at risk for developing a dopamine disorder described herein, the clathrin nanoparticle is further linked to a neurotrophin. In some embodiments of any of the methods for treating a human subject having or at risk for developing a dopamine disorder described herein, the clathrin nanoparticle comprises a clathrin cage. In some embodiments of any of the methods for treating a human subject having or at risk for developing a dopamine disorder described herein, the clathrin nanoparticle consists of a clathrin triskelion. In some embodiments of any of the methods for treating a human subject having or at risk for developing a dopamine disorder described herein, the clathrin nanoparticle comprises a complex consisting of 1 to 3 clathrin heavy chains. In some embodiments of any of the methods for treating a human subject having or at risk for developing a dopamine disorder described herein, the DAT targeting agent comprises a dopamine re-uptake inhibitor or an anti-DAT antibody. In some embodiments of any of the methods for treating a human subject having or at risk for developing a dopamine disorder described herein, the dopamine reuptake inhibitor is Vanoxerine. In some embodiments of any of the methods for treating a human subject having or at risk for developing a dopamine disorder described herein, the neurotrophin comprises BDNF, NGF, NT-3, NT-4, NT-6, GDNF, NTN, PSPN, ARTN, CNTF, or LIF. In some embodiments of any of the methods for treating a human subject having or at risk for developing a dopamine disorder described herein, the DAT targeting agent is linked to the clathrin nanoparticles by conjugation. In some embodiments of any of the methods for treating a human subject having or at risk for developing a dopamine disorder described herein, the DAT targeting. In some embodiments of any of the methods for treating a human subject having or at risk for developing a dopamine disorder described herein, the neurotrophin is linked to the clathrin nanoparticles by conjugation. In some embodiments of any of the methods for treating a human subject having or at risk for developing a dopamine disorder described herein, the neurotrophin is conjugated to the clathrin nanoparticles via PEG. In some embodiments of any of the methods for treating a human subject having or at risk for developing a dopamine disorder described herein, the DAT targeting agent and the neurotrophin are linked to the same heavy chain of the clathrin triskelion. In some embodiments of any of the methods for treating a human subject having or at risk for developing a dopamine disorder described herein, the DAT targeting agent and the neurotrophin are linked to different heavy chains of the clathrin triskelion. In some embodiments of any of the methods for treating a human subject having or at risk for developing a dopamine disorder described herein, a clathrin heavy chain of the clathrin triskelion is linked to 1 to 5 molecules of the DAT targeting agent. In some embodiments of any of the methods for treating a human subject having or at risk for developing a dopamine disorder described herein, a clathrin heavy chain of the clathrin triskelion is linked to 1 to 5 molecules of the neurotrophin. In some embodiments of any of the methods for treating a human subject having or at risk for developing a dopamine disorder described herein, a clathrin heavy chain of the clathrin triskelion is linked to 1 to 5 molecules of the DAT targeting agent and 1 to 5 molecules of the neurotrophin. In some embodiments of any of the methods for treating a human subject having or at risk for developing a dopamine disorder described herein, the clathrin heavy chain of the clathrin triskelion is linked to one molecule of BDNF and four molecules of Vanoxerine. In some embodiments of any of the methods for treating a human subject having or at risk for developing a dopamine disorder described herein, the clathrin heavy chain of the clathrin triskelion is linked to one molecule of BDNF and three molecules of an anti-DAT antibody. one of the 1 to 3 clathrin heavy chains is linked to 1 to 5 molecules of the DAT targeting agent. In some embodiments of any of the methods for treating a human subject having or at risk for developing a dopamine disorder described herein, one of the 1 to 3 clathrin heavy chains is linked to 1 to 5 molecules of the neurotrophin. In some embodiments of any of the methods for treating a human subject having or at risk for developing a dopamine disorder described herein, one of the 1 to 3 clathrin heavy chains is linked to 1 to 5 molecules of the DAT targeting agent and 1 to 5 molecules of the neurotrophin. In some embodiments of any of the methods for treating a human subject having or at risk for developing a dopamine disorder described herein, the dopamine disorder is a neurodegenerative disease associated with loss of dopaminergic neurons. In some embodiments of any of the methods for treating a human subject having or at risk for developing a dopamine disorder described herein, the neurodegenerative disease associated with loss of dopaminergic neurons is Parkinson's disease, Huntington's disease, Alzheimer's disease, or ALS. In some embodiments of any of the methods for treating a human subject having or at risk for developing a dopamine disorder described herein, the dopamine disorder is ADHD, depression, or schizophrenia. In some embodiments of any of the methods for treating a human subject having or at risk for developing a dopamine disorder described herein, the dopamine disorder is a dopamine-driven addiction. In some embodiments of any of the methods for treating a human subject having or at risk for developing a dopamine disorder described herein, the dopamine-driven addiction is methamphetamine use disorder, cocaine addiction, opioid addiction, sex addiction, food addiction, or gambling addiction. In some embodiments of any of the methods for treating a human subject having or at risk for developing a dopamine disorder described herein, the composition is delivered intranasally or intravenously.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 70% (e.g., at least 80%, 90% or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the Needleman and Wunsch ((1970) J. Mol. Biol. 48:444-453) algorithm, which has been incorporated into the GAP program in the GCG software package (available at gcg.com), using either a Blossum 62 matrix, a PAM250 matrix, a NWSgapdna. CMP matrix. In some embodiments, the percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

### DESCRIPTION OF DRAWINGS

FIGS. 1A-1E: Structure, size, and specificity of DAT-ab-BDNF-clathrin nanoparticles (CNPs). (A) A diagram of a clathrin triskelion (CT) with BDNF and anti-dopamine transporter (DAT) antibody (DAT-ab) conjugated to the CT heavy chains via PEGs. (B-C) Quantification of hydrodynamic radius (RH) of unconjugated CTs (B; mean=16.8±5.6 nm) and CNPs (C; mean=42.13±14.8 nm), as determined by dynamic light scattering. (D-E) Fluorescent images of mouse striatal slices labeled in vitro with fluorescently-labeled CNPs conjugated with DAT-ab (D) or conjugated with IgG (E).

FIGS. 2A-2I: DAT-ab-BDNF-CNP brain distribution following intranasal (i.n.) delivery. (A-C) Fluorescent images of the mouse striatum (STR; A), substantia nigra (SN; B), and frontal cortex (FC; C) 4 hours after i.n. delivery of fluorescent NSH-fluorescein labeled CNPs (green). (D-F) Quantification of radioactive ³H-DAT-ab-BDNF-CNPs concentrations in the STR (D), SN (E), and FC (F) of inducible HIV-1 Tat transgenic (iTat) mice 4 hours and 6 hours after intranasal delivery. ³H-DAT-ab-BDNF-CNPs concentrations were determined using scintillation counting methods. Data are represented as the mean % of the injected dose per gram (% ID/g) of tissue (n=3/group). Error bars represent S.D. (G-I) Iron staining of rat STR (G), SN (H), and FC (I) 4 hours after delivery of iron oxide labeled DAT-ab-BDNF-CNPs. Scale bar represents 100 μm.

FIGS. 3A-3C: DAT-ab-BDNF-CNPs targeted DATs in vivo. (A-C) Fluorescent labeling of DAT (red, A), i.n. delivered NSH-fluorescein labeled DAT-ab-BDNF-CNPs (green, B), and overlay (C). The scale bar is 25 um.

FIGS. 4A-4D: DAT-ab-BDNF-CNPs rescue TH+ fibers from Tat-induced toxicity in the striatum. (A-C) Fluorescent staining for tyrosine hydroxylase (TH) (red) in striatal sections of Tat+ mice receiving intraperitoneal (i.p.) doxycycline (Dox) and i.n. treatments of saline (Sal; A) or DAT-ab-BDNF-CNPs (B). A global control group (Tat−) received only saline. (D) Quantification of TH+ fiber density in the striatum of Tat+ mice receiving DAT-ab-BDNF-CNPs versus control. TH+ fiber density was higher in DAT-ab-BDNF-CNP-treated versus saline-treated Tat+ mice. TH+ fiber densities are expressed as the mean percentage of Sal-treated controls. Error bars represent S.E.M. (n=3-4 per group; *<0.05).

FIGS. 5A-5D: DAT-ab-BDNF-CNPs rescue TH+ fibers from METH-induced toxicity in the striatum. (A-C) Fluorescent staining for TH+ fibers (red) in striatal sections of Tat− mice receiving i.p. Sal, i.p. D-METH HCl (4 mg/kg), and i.n. treatments of Sal (A) or DAT-ab-BDNF-CNPs (B). A global control group received only saline (C). (D) Quantification of TH+ fiber density in the striatum of METH-treated mice receiving DAT-ab-BDNF-CNPs versus control. TH+ fiber density was higher in DAT-ab-BDNF-CNP-treated versus saline-treated mice receiving METH. TH+ fiber densities are expressed as the mean percentage of Sal-treated controls. Error bars represent S.E.M. (n=4-5 per group; **<0.01).

FIGS. 6A-6D: DAT-ab-BDNF-CNPs rescue TH+ fibers from METH- and Tat-induced toxicity in the striatum. (A-C) Fluorescent staining for TH+ fibers in striatal sections of Tat+ mice receiving i.p. Dox, i.p. D-METH HCl (4 mg/kg), and i.n. Sal (A) or CNP (B). A global control group (Tat−) received only saline (C). (D) Quantification of TH+ fiber density in the striatum of Tat+ and METH-treated mice receiving DAT-ab-BDNF-CNPs versus control. TH+ fiber density was higher in DAT-ab-BDNF-CNP-treated versus Sal-treated Tat+ mice receiving METH. TH+ fiber densities are expressed as the percentage of Sal-treated, Tat− controls. Data are represented as the mean percentage. Error bars represent S.E.M. (n=4 per group; *<0.05).

FIGS. 7A-7B: FIG. 7A is a graphical representation of GBR and BDNF linked triskelia binding to TrkB and DAT. FIG. 7B is a transmission electron microscopy image of isolated triskelia.

FIGS. 8A-8G: Affinity of GBR-CNPs to rat brain. (A) MALDI-TOF mass spectra of PEGylated GBR12909 (GBR) ligands. Molecular weight=4 kDa. (B) Binding curve of ¹⁴C-GBR-CNPs to rat striatal slices. Striatal rat slices were incubated for 120 minutes with 10 different concentrations of ¹⁴C-GBR-CNPs (0.06 to 158.7 nM). Kd value=3. (C-F) Autoradiograms of rat caudate-putamen (CPu) and cerebellum (CER) in the presence of 2 nM ¹⁴C-GBR-CNP (C, D) or 1 μM of cold GBR (E, F). (G) Quantification of CNP specific binding (total binding—nonspecific binding) in the CPu (L, lateral, and M, medial), nucleus accumbens (NAc), and CER. Data are presented as mean values±SD. The striatum-to-cerebellum ratio was 2.3.

FIGS. 9A-9C: GBR-CNPs specifically target DAT in vivo. (A) Quantification of GBR-CNPs concentration in the rat brain and blood 45 min., 90 min., and 180 min., post intravenous (i.v.) injection of ¹⁵³GdDTPA-GBR-CNPs (n=4 per time point). Data represent the mean percentages of the injected dose per gram of tissue (% ID/g). (B-C) Fluorescent images of rat CPu (B) and CER (C) 90 minutes after i.n. delivery of FITC-GBR-BDNF-CNPs (green). CNPs were detected in the rat CPu but not CER. Scale bar represents 100 μm.

FIG. 10: Setup for GRB-BDNF-CNP biodistribution and pharmacokinetics studies.

FIG. 11: Setup for assessing the effects of GBR-BDNFs-CNPs on cognitive and motor deficits in rats exposed to toxic binge doses of METH. OF=Open Field test.

FIGS. 12A-12C: Clathrin nanoparticles rescue motor behavior from METH-induced toxicity. (A-C) Quantification of forelimb strength (A) and speed (B) and distance traveled (C) in the Open Field (OF) test of mice receiving i.p. D-METH HCl (4 mg/kg) and i.n. saline or GBR-BDNF-CNPs. *p<0.05 and **p<0.01. Error bars are S.E.M. (n=8-9 per group).

FIGS. 13A-13C: BDNF-CNPs improved novel object recognition. (A) Quantification of novel object recognition in mice receiving saline; BDNF-CNPs with 0.3 mg/kg of BDNF and 2.4 mg/kg of CTs; or BDNF-CNPs with 0.15 mg/kg of BDNF and 1.2 mg/kg of CTs. (n=5-10 mice; *p<0.05, Tukey's HSD). (B-C) Image representing the amount of time spent by mice in areas of the test chamber.

FIG. 14: Setup for assessing effect of GBR-BDNFs-CNPs on context-, cue-, or METH-induced METH seeking behavior in rats.

### DETAILED DESCRIPTION

Provided herein are compositions and methods for treating dopamine disorders. In one aspect, provided herein are compositions comprising a dopamine active transporter (DAT) targeting agent linked to a clathrin nanoparticle. In another aspect, provided herein are methods for treating a human subject having or at risk for developing a dopamine disorder comprising administering to the human subject a composition comprising a DAT targeting agent linked to a clathrin nanoparticle. In some embodiments of any of the above aspects, the clathrin nanoparticle is further linked to a neurotrophin.

### Clathrin Nanoparticles

The clathrin nanoparticles described herein can include clathrin cages, clathrin triskelia, or one or more clathrin heavy chains. See, e.g., U.S. Pat. No. 7,393,924 and US2014/0288192. A clathrin triskelion can consist of three clathrin heavy chains (CHC), and zero to three clathrin light chains (CLC) interacting at their C-termini, preferably where each heavy chain (about 190 kDa) has a light chain (about 25 kDa) bound to it. The three heavy chains provide the structural backbone of the clathrin cage, and the three light chains are thought to regulate the formation and disassembly of a clathrin cage. The CHC leg can be divided up into three regions: a proximal region that is close to the central vertex and is where the light chain attaches, a distal region, and the N-terminal domain at the end of the leg. An exemplary sequence of the human CHC is shown below (SEQ ID NO: 1):

| | |
|---|---|
| 1 | MAQILPIRFQ EHLQLQNLGI NPANIGFSTL TMESDKFICI REKVGEQAQV VIIDMNDPSN |
| 61 | PIRRPISADS AIMNPASKVI ALKAGKTLQI FNIEMKSKMK AHTMTDDVTF WKWISLNTVA |
| 121 | LVTDNAVYHW SMEGESQPVK MFDRHSSLAG CQIINYRTDA KQKWLLLTGI SAQQNRVVGA |
| 181 | MQLYSVDRKV SQPIEGHAAS FAQFKMEGNA EESTLFCFAV RGQAGGKLHI IEVGTPPTGN |
| 241 | QPFPKKAVDV FFPPEAQNDF PVAMQISEKH DVVFLITKYG YIHLYDLETG TCIYMNRISG |
| 301 | ETIFVTAPHE ATAGIIGVNR KGQVLSVCVE EENIIPYITN VLQNPDLALR MAVRNNLAGA |
| 361 | EELFARKFNA LFAQGNYSEA AKVAANAPKG ILRTPDTIRR FQSVPAQPGQ TSPLLQYFGI |
| 421 | LLDQGQLNKY ESLELCRPVL QQGRKQLLEK WLKEDKLECS EELGDLVKSV DPTLALSVYL |
| 481 | RANVPNKVIQ CFAETGQVQK IVLYAKKVGY TPDWIFLLRN VMRISPDQGQ QFAQMLVQDE |
| 541 | EPLADITQIV DVFMEYNLIQ QCTAFLLDAL KNNRPSEGPL QTRLLEMNLM HAPQVADAIL |
| 601 | GNQMFTHYDR AHIAQLCEKA GLLQRALEHF TDLYDIKRAV VHTHLINPEW LVNYFGSLSV |
| 661 | EDSLECLRAM LSANIRQNLQ ICVQVASKYH EQLSTQSLIE LFESEKSFEG LFYFLGSIVN |
| 721 | FSQDPDVHFK YIQAACKTGQ IKEVERICRE SNCYDPERVK NFLKEAKLTD QLPLIIVCDR |
| 781 | FDFVHDLVLY LYRNNLQKYI EIYVQKVNPS RLPVVIGGLL DVDCSEDVIK NLILVVRGQF |
| 841 | STDELVAEVE KRNRLKLLLP WLEARIHEGC EEPATHNALA KIYIDSNNNP ERFLRENPYY |
| 901 | DSRVVGKYCE KRDPHLACVA YERGQCDLEL INVCNENSLF KSLSRYLVRR KDPELWGSVL |
| 961 | LESNPYRRPL IDQVVQTALS ETQDPEEVSV TVKAFMTADL PNELIELLEK IVLDNSVFSE |
| 1021 | HRNLQNLLIL TAIKADRTRV MEYINRLDNY DAPDIANIAI SNELFEEAFA IFRKFDVNTS |
| 1081 | AVQVLIEHIG NLDRAYEFAE RCNEPAVWSQ LAKAQLQKGM VKEAIDSYIK ADDPSSYMEV |
| 1141 | VQAANTSGNW EELVKYLQMA RKKARESYVE TELIFALAKT NRLAELEEFI NGPNNAHIQQ |
| 1201 | VGDRCYDEKM YDAAKLLYNN VSNFGRLAST LVHLGEYQAA VDGARKANST RTWKEVCFAC |
| 1261 | VDGKEFRLAQ MCGLHIVVHA DELEELINYY QDRGYFEELI TMLEAALGLE RAHMGMFTEL |
| 1321 | AILYSKFKPQ KMREHLELFW SRVNIPKVLR AAEQAHLWAE LVFLYDKYEE YDNAIITMMN |
| 1381 | HPTDAWKEGQ FKDIITKVAN VELYYRAIQF YLEFKPLLLN DLLMVLSPRL DHTRAVNYFS |
| 1441 | KVKQLPLVKP YLRSVQNHNN KSVNESLNNL FITEEDYQAL RTSIDAYDNF DNISLAQRLE |
| 1501 | KHELIEFRRI AAYLFKGNNR WKQSVELCKK DSLYKDAMQY ASESKDTELA EELLQWFLQE |
| 1561 | EKRECFGACL FTCYDLLRPD VVLETAWRHN IMDFAMPYFI QVMKEYLTKV DKLDASESLR |
| 1621 | KEEEQATETQ PIVYGQPQLM LTAGPSVAVP PQAPFGYGYT APPYGQPQPG FGYSM |

Residues 1-479 can also be referred to as the N-terminal globular domain region, where residues 24-330 include seven repeat regions, residues 331-394 are a flexible linker region, and residues 449-465 are the binding site for the uncoating ATPase.

Residues 457-507 are a region involved in spindle location and interaction with TACC3. Residues 524-1675 can be referred to as the heavy chain arm region, with residues 524-634 being the distal segment region, and residues 639-1675 being the proximal segment region. Within the heavy chain arm region, residues 1213-1522 is a region involved in binding clathrin light chain, and residues 1550-1675 is the trimerization domain region (See, e.g., Wakeham et al. The EMBO Journal 22(19): 4980-4990, 2003). Additional domain characterizations can be found at least at Greene et al. Traffic 1:69-75, 2000.

Humans have two clathrin light chains (CLCa and CLCb), and both can associate with CHC. An exemplary sequence of human CLCa is shown below (SEQ ID NO: 2):

| | |
|---|---|
| 1 | MAELDPFGAP AGAPGGPALG NGVAGAGEED PAAAFLAQQE SEIAGIENDE AFAILDGGAP |
| 61 | GPQPHGEPPG GPDAVDGVMN GEYYQESNGP TDSYAAISQV DRLQSEPESI RKWREEQMER |
| 121 | LEALDANSRK QEAEWKEKAI KELEEWYARQ DEQLQKTKAN NRVADEAFYK QPFADVIGYV |
| 181 | TNINHPCYSL EQAAEEAFVN DIDESSPGTE WERVARLCDF NPKSSKQAKD VSRMRSVLIS |
| 241 | LKQAPLVH |

An exemplary sequence of human CLCb is shown below (SEQ ID NO: 3):

| | |
|---|---|
| 1 | MADDFGFFSS SESGAPEAAE EDPAAAFLAQ QESEIAGIEN DEGFGAPAGS HAAPAQPGPT |
| 61 | SGAGSEDMGT TVNGDVFQEA NGPADGYAAI AQADRLTQEP ESIRKWREEQ RKRLQELDAA |
| 121 | SKVTEQEWRE KAKKDLEEWN QRQSEQVEKN KINNRIADKA FYQQPDADII GYVASEEAFV |
| 181 | KESKEETPGT EWEKVAQLCD FNPKSSKQCK DVSRLRSVLM SLKQTPLSR |

The clathrin triskelia can be purified natural triskelia or recombinant triskelia. Methods of purifying triskelia or generating recombinant triskelia are known in the art. Exemplary methods of purifying triskelia can be found at Keen et al. Cell 16:303-312, 1979 and Kirchhausen and Harrison, Cell 23(3):755-761, 1981. Triskelia of the present disclosure can be those purified from e.g. bovine brain. Recombinant triskelia can be generated by co-expressing clathrin heavy and light chains in e.g. insect cells, followed by purification. The recombinant heavy and light chains can form triskelia, which in turn can spontaneously assemble into cages (e.g., if transferred into low-salt conditions). Alternatively, recombinant heavy chain and light chains can be expressed separately, and reconstituted. For example, purified clathrin light chain expressed in E. coli can be added to purified heavy chain expressed in insect cells (See, e.g., Rapoport et al. Molecular Biology of the Cell 19(1):405-413, 2008).

Clathrin cages are geodesic assemblies of clathrin triskelia and can include varying numbers of triskelia with different geometries. For example, a clathrin cage can have 28, 36, or 60 triskelia, and has a tetrahedral symmetry, hexagonal shaped D6 symmetry, or football shaped icosahedral symmetry, respectively (See, e.g., Royle, Cell Mol Life Sci 63(16): 1823-1832, 2006).

Clathrin cages can be generated by self-assembly of purified or recombinant triskelia. Below pH 6.5, purified clathrin triskelia self-assemble in vitro into polyhedral cages without vesicles, but typically only form cages at physiological pH in the presence of stoichiometric quantities of purified AP-1 or AP-2 adaptor molecules or the neuron-specific assembly proteins AP-180 and auxilin. The adaptors can both trigger clathrin self-assembly and co-assemble with clathrin.

Alternatively, fragments of triskelia can be combined to form cages. For example, the terminal and distal domains of the heavy chain (TDD, residues 1-1074) and hub domain (residues 1074-1675) of the heavy chain occupied by clathrin light chain (Hub/Lcb) can be expressed (e.g., recombinantly), and assembled under conditions similar to those used for self-assembly of isolated clathrin triskelia (See, e.g., Greene et al. Traffic 1:69-75, 2000). The recombinant hub domains are trimeric structures that reproduce the central portion of the three-legged clathrin triskelion, extending from the vertex to the bend in each leg, comprising the binding sites for clathrin light chain subunits. Without light chain subunits, recombinant hub domains can self-assemble reversibly at physiological pH, while hub domains with bound light chains only self-assemble below pH 6.5, similar to purified triskelion, as light chain subunits inhibit hub assembly at physiological pH. The clathrin cages can have a diameter of at least about 10 (e.g., at least about 20, 30, 40, 50, 60, 70, 80, 90, or 100) nanometers.

The compositions provided herein can include clathrin nanoparticles comprising a clathrin cage made up of a plurality of clathrin triskelia, where one or more heavy chains of a triskelion are linked to a DAT targeting agent and/or a neurotrophin as described herein. The clathrin nanoparticles can also comprise a single clathrin triskelion, where one or more heavy chains of the triskelion are linked to a DAT targeting agent and/or a neurotrophin as described herein. The DAT targeting agent and the neurotrophin can be linked to the same or different heavy chains within a triskelion. A heavy chain of a triskelion can be linked to up to 5 molecules (e.g., 1, 2, 3, or 4 molecules) of the DAT targeting agent, and or up to 5 molecules (e.g., 1, 2, 3, or 4 molecules) of the neurotrophic factor. In some instances, the present composition comprises on average one molecule of a neurotrophic factor and 4 molecules of a DAT targeting agent linked to one CHC of a triskelion.

In another example, the clathrin nanoparticle can comprise one or more isolated or recombinant CHC proteins (e.g., full length proteins or fragments thereof), and the CHC proteins are linked to a DAT targeting agent and/or a neurotrophin. Fragments of the CHC proteins contemplated herein can include one or more suitable portions of the full length protein, such as the hub domain (e.g., the trimerization domain), the N-terminal domain, or the hub domain attached to the N-terminal domain.

For example, the composition can consist of (1) one isolated or recombinant CHC protein or portions thereof linked to a DAT targeting agent and a neurotrophin; or (2) two or more isolated or recombinant CHC proteins or portions thereof, where the DAT targeting agent and the neurotrophin are linked to the same or different CHC proteins. In some instances, the CHC proteins can be bound by one CLC.

The sequences used in the present methods and compositions can be identical to the reference sequences provided herein, or can be at least 70% identical (e.g., at least 75%, 80%, 82%, 84%, 86%, 88%, 90%, 91%, 92%, 93%, 94%, 95%, 96%, 97%, 98%, or 99% identical) to the reference sequences. For examples, recombinant CHC proteins that are at least 70% identical (e.g., at least 75%, 80%, 82%, 84%, 86%, 88%, 90%, 91%, 92%, 93%, 94%, 95%, 96%, 97%, 98%, or 99% identical) to SEQ ID NO: 1, and/or recombinant CLC proteins that are at least 70% identical (e.g., at least 75%, 80%, 82%, 84%, 86%, 88%, 90%, 91%, 92%, 93%, 94%, 95%, 96%, 97%, 98%, or 99% identical) to SEQ ID NO: 2 or 3, or a portion thereof, are contemplated herein.

Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 70% (e.g., at least 80%, 90% or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the Needleman and Wunsch ((1970) J. Mol. Biol. 48:444-453) algorithm, which has been incorporated into the GAP program in the GCG software package (available at gcg.com), using either a Blossum 62 matrix, a PAM250 matrix, a NWSgapdna. CMP matrix. In some embodiments, the percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The average size of the clathrin nanoparticles provided herein can be any size that is suitable for crossing the blood brain barrier. For example the nanoparticles can have a cross-sectional diameter of between 1-100 nm (e.g., about 10-80, 20-60, or 30-50 nm). The clathrin nanoparticles provided herein can further include a fluorescent label, a radiolabel (e.g., ¹⁴C, ³H, and ¹⁵³Gd), or an ion-oxide label, which may be helpful in detecting the nanoparticles in vivo.

### Dopamine Transporter (DAT) Targeting Agent

Dopamine transporter (or dopamine active transporter, or DAT) is a plasma membrane protein that actively translocates dopamine from the extracellular space into the presynaptic neuron. A DAT targeting agent provided herein can modulate the activity of DAT thereby modulates the dynamics of dopamine neurotransmission. DAT targeting agents contemplated herein include but are not limited to dopamine reuptake inhibitors (DRIs), anti-DAT antibodies, and DAT binding ligands (e.g., Benztropine analogs).

### Dopamine Reuptake Inhibitor (DRIs)

DRIs can block the action of the dopamine transporter (DAT) thereby inhibit reuptake of dopamine. Dopamine reuptake inhibitors can result in increased extracellular concentrations of dopamine and increase in dopaminergic neurotransmission. DRIs can be selective for DAT over other monoamine transporters, and can have varying degrees of selectivity for dopamine over other neurotransmitters such as norepinephrine or serotonin. Some DRIs can have effects on both dopamine and norepinephrine. Any DRIs described herein or known in the art are contemplated for the compositions and methods of the present disclosure. Exemplary DRIs include: 4-Hydroxy-1-methyl-4-(4-methylphenyl)-3-piperidyl 4-methylphenyl ketone, Altropane (O-587), Amfonelic acid (WIN 25978), Amineptine, BTCP (GK-13), 3C-PEP, DBL-583, Difluoropine (O-620), GBR-12783, GBR-12935, GBR-13069, GBR-13098, GYKI-52895, Iometopane (β-CIT, RTI-55), Methylphenidate, Ethylphenidate, Modafinil, Armodafinil, RTI-229, and Vanoxerine (GBR-12909). Additional DRIs include drafinil, Amantadine, Benztropine, Bupropion, Fluorenol, Ketamine, Medifoxamine, Metaphit, Rimcazole, Sertraline, St. John's Wort, Venlafaxine, Chaenomeles speciose, 3-Methoxyphencyclidine, 4-Methoxyphencyclidine, and Oroxylin A.

Vanoxerine (GBR-12909, 1-[2-[bis(4-fluorophenyl) methoxy]ethyl]-4-(3-phenylpropyl) piperazine) is a piperazine derivative. It can bind to the target site on the DAT and inhibits dopamine reuptake. The structure of Vanoxerine is shown below:

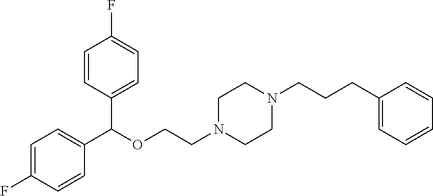 [SMILES: Fc1ccc(C(OCCN2CCN(CCCc3ccccc3)CC2)c2ccc(F)cc2)cc1]

In some instances, the present disclosure comprises delivering DRIs (e.g., GBR) to a subject via nasal administration of nanoparticles. The dose of the DRI delivered can be about 0.1 mg/kg to about 5 mg/kg (e.g., about 0.1 to about 4, about 0.1 to about 3, about 0.1 to about 2, about 0.1 to about 1, about 0.1 to about 0.5 about 0.5 to about 5, about 0.5 to about 4, about 0.5 to about 3, about 0.5 to about 2, about 0.5 to about 1, about 1 to about 5, about 1 to about 4, about 1 to about 3, about 1 to about 2, about 2 to about 5, about 2 to about 4, about 2 to about 3, about 3 to about 5, about 3 to about 4, or about 4 to about 5 mg/kg).

### Anti-DAT Antibody

The DAT targeting agent linked to clathrin nanoparticles can be an anti-DAT antibody. The anti-DAT antibody can bind specifically to DAT, or can bind to other targets in addition to DAT. Clathrin nanoparticles linked to anti-DAT antibodies can be used to target the nanoparticles to specific brain regions, such as but not limited to the striatum and the substantia nigra. One to five molecules (e.g. 1, 2, 3, 4, or 5) of anti-DAT antibodies can be linked to a clathrin triskelion. In some instances, three molecules of an anti-DAT antibody and one molecule of BDNF are linked to a triskelion.

Usage of the term “antibody” in this disclosure is meant to cover a whole antibody (as opposed to a minibody, nanobody or antibody fragment), a bispecific antibody, a tertravalent antibody, a multispecific antibody, a minibody, a nanobody, and antibody fragments. In certain instances, the heavy chain constant region of the anti-DAT antibody is a human IgG1, human IgG2, human IgG3, or human IgG4 constant region. In certain instances, the light constant region is a human kappa constant region. In other instances, the light constant region is a human lambda constant region. The anti-DAT antibodies can be a human, humanized, chimeric, or single domain antibody. The anti-DAT antibodies can also be a Fv, a scFv, an sc(Fv)2, a diabody, a nanobody, an Fab, or an F(ab′)2.

Any anti-DAT antibodies known in the art can be used in the methods and compositions contemplated herein. Exemplary anti-DAT antibodies include ab111468 (Abcam), PA1-4657 (ThermoFisher), AB1591P or MABN669 (EMD Millipore), GTX133152 (GeneTex), sc-32258 (Santa Cruz Biotechnology). The anti-DAT antibodies linked to clathrin nanoparticles described herein can also be any anti-DAT antibodies that competes with any anti-DAT antibodies known in the art (e.g., any of the exemplary anti-DAT antibodies described herein). Suitable anti-DAT antibodies can also be produced based on methods known in the art.

General methods for making antibodies, e.g., monospecific, polyclonal, or monoclonal antibodies, are known in the art. For monoclonal antibodies, the process involves obtaining antibody-secreting immune cells (lymphocytes) from the spleen of a mammal (e.g., mouse) that has been previously immunized with the antigen of interest (e.g., a peptide antigen as described herein) either in vivo or in vitro. The antibody-secreting lymphocytes are then fused with myeloma cells or transformed cells that are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells can be found in Kohler and Milstein, Nature 256:495 (1975).

Procedures for raising polyclonal antibodies are also known. Typically, such antibodies can be raised by administering the protein or polypeptide of the present invention subcutaneously to New Zealand white rabbits that have first been bled to obtain pre-immune serum. The antigens can be injected, e.g., at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized, e.g., with pentobarbital 150 mg/Kg IV. This and other procedures for raising polyclonal antibodies are disclosed in E. Harlow et al., editors, Antibodies: A Laboratory Manual (1988).

Variants of any of the anti-DAT antibodies described herein are also contemplated. Methods of generating antibody variants (e.g., comprising amino acid substitutions) are well known in the art. These methods include, but are not limited to, preparation by site-directed (or oligonucleotide-mediated) mutagenesis, PCR mutagenesis, and cassette mutagenesis of a prepared DNA molecule encoding the antibody or any portion thereof (e.g., a framework region, a CDR, a constant region). Site-directed mutagenesis is well known in the art (see, e.g., Carter et al., Nucl. Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nucl. Acids Res. 17:723-733 (1989). Another method for preparing sequence variants, cassette mutagenesis, is based on the technique described by Wells et al., Gene, 34:315-323 (1985).

Additional ligands that bind to DAT are also contemplated herein. Benztropine analogs, for example, can be linked to the clathrin nanoparticles as a DAT targeting agent (See, Desai et al. J Neuroscience 25(8):1889-1893, 2005).

### Neurotrophins

Neurotrophins or neurotrophic factors are a family of proteins that can induce the survival, development and function of neurons (e.g., sensory and sympathetic neurons) in both the peripheral and central nervous systems. Neurotrophins can activate one or more of the three members of the tropomyosin-related kinase (Trk) family of receptor tyrosine kinases (TrkA, TrkB, and TrkC). In addition, neurotrophins activate p75 neurotrophin receptor (p75NTR), a member of the tumor necrosis factor receptor superfamily. Through Trk receptors, neurotrophins activate Ras, phosphatidyl inositol-3 (PI3)-kinase, phospholipase C-gamma1 and signaling pathways controlled through these proteins, such as the MAP kinases. Activation of p75NTR results in activation of the nuclear factor-kappaB (NF-kappaB) and Jun kinase as well as other signaling pathways. Continued presence of the neurotrophins is required in the adult nervous system, where they control synaptic function and plasticity, and sustain neuronal survival, morphology and differentiation.

Neurotrophins suitable for linking to the clathrin nanoparticles described herein include but are not limited to: nerve growth factor (NGF), brain-derived neurotrophic factor (BDNF), neurotrophin-3 (NT-3) and neurotrophin-4 (NT-4), NT-6, NTN, PSPN, ARTN, CNTF, and LIF. Additional proteins that regulate neuronal survival and/or other aspects of neuronal development are also contemplated herein, such as but are not limited to glial cell-derived neurotrophic factor.

In some instances, the neurotrophin linked to clathrin nanoparticles described herein is BDNF. BDNF proteins can be unstable and do not easily cross the BBB (See. e.g., Gilmore et al. J Neuroimmune Pharmacol 3(2): p. 83-94, 2008). BDNF has a short in vivo half-life (<5 min) and poor pharmacokinetic profile, which makes treatment with BDNF difficult. However, agents such as antidepressants and mood stabilizers that can increase BDNF levels act on different sites and have multiple side effects (See, e.g., Bhaskar et al. Part Fibre Toxicol. 7: p. 3, 2010). Without wishing to be bound by theory, linking BDNF to clathrin nanoparticles as described herein allows BDNF to be delivered across the BBB.

### Linking Methods

The clathrin nanoparticles can be linked to the DAT targeting agents and/or the neurotrophins described herein in a number of ways, such as but not limited to, conjugation, cross-linking, covalent bonding, and biotin-avidin interaction. In some embodiments, the clathrin nanoparticles are linked to the DAT targeting agents or the neurotrophins via functional groups. In some embodiments, the functional groups are part of a polymer. The polymer can be a synthetic polymer, such as, but not limited to, polyethylene glycol or silane, natural polymers, or derivatives of either synthetic or natural polymers or a combination thereof. Hydrophilic polymers are useful for as a linking polymer. The polymer can include polysaccharides and derivatives, including dextran, pullanan, carboxydextran, carboxmethyl dextran, and/or reduced carboxymethyl dextran. The polymers can be functionalized (e.g., including amino, carboxyl, or other reactive groups) for attaching the DAT targeting agents, neurotrophins, or any additional desired moieties to the clathrin nanoparticles. In some instances, the cysteines of a clathrin triskelion can be used to attach PEGs, which are then used to crosslink the DAT targeting agent or the neurotrophin. Exemplary methods of pegylating nanoparticles can be found at e.g., U.S. Pat. Nos. 7,291,598; 5,145,684; 6,270,806; and 7,348,030.

Avidin or streptavidin can be attached to the clathrin nanoparticles for use with a biotinylated binding moiety, such as an oligonucleotide or polypeptide. See e.g., Shen et al., Bioconjug. Chem., 1996, 7(3):311-6. Similarly, biotin can be attached to the nanoparticles for use with an avidin-labeled binding moiety. Low molecular weight compounds can be separated from the nanoparticles by ultra-filtration, dialysis, magnetic separation, or other means. The unreacted DAT targeting agents or neurotrophins can be separated from the ligand-nanoparticle conjugates, e.g., by size exclusion chromatography. One, two, or all three of the heavy chains of a triskelion can be linked to a DAT targeting agent or to a neurotrophin. The DAT targeting agent and the neurotrophin can be linked to the same or different heavy chains of a triskelion.

### Methods of Treatment

The present disclosure provides methods for treating dopamine disorders, such as dopamine-driven addictions and neurodegenerative diseases associated with the loss of dopaminergic neurons. Generally, the methods include administering a therapeutically effective amount of the composition described herein, to the subject who is in need of, or who has been determined to be in need of, such treatment. The composition comprises a dopamine active transporter (DAT) targeting agent linked to a clathrin nanoparticle. In some embodiments, the clathrin nanoparticle is further linked to a neurotrophin.

### Subject Selection

Dopamine-driven addictions can include addictions to substances or activities that involve increased extracellular dopamine levels, such as but not limited to: drug addiction, sex addiction, food addiction, and gambling addiction. Methamphetamine use disorder (MUD) and opioid addiction are examples of drug addiction. Additional dopamine disorders include schizophrenia, ADHD, and depression. Neurodegenerative diseases associated with the loss of dopaminergic neurons can include Alzheimer's Disease, Parkinson's Disease, Amyotrophic Lateral Sclerosis, and Huntington's Disease. Additional dopamine-associated disorders can be found e.g., in Barbeau, C. M. A. Journal, 103:824-832, 1970. Loss of dopaminergic neurons may be detected in one or more parts of the subject's brain, e.g. in substantia nigra and/or nucleus accumbens. For example, Parkinson's disease is a degenerative condition causing tremor and motor impairment, and is caused by a loss of dopamine-secreting neurons in the substantia nigra.

Methamphetamine use disorder (MUD) represents major chronic health problems in the US and abroad with significant neuropsychiatric, cognitive and socioeconomic consequences. Studies have demonstrated the role of neuroinflammation and neurodegeneration [64-70] in the pathogenesis of MUD (1-7). METH is neurotoxic and can activate microglial cells; increase inflammatory cytokines, glutamate or free radicals; decrease neuroprotective/neurotrophic factors; and reduce neurogenesis and synaptogenesis (3,4). Thus, METH can have a deleterious impact on brain structure and function. MRI studies consistently showed striatal structural and functional abnormalities associated with deficits in cognitive and motor functions (1, 8-14). PET studies reported dopamine transporter (DAT) density reductions in patients with MUD (15-19). Therefore, long-term METH use is associated with decreased DA function and neurodegeneration and new pharmaceuticals for treatment of both METH addiction and toxicity are required.

Patients with MUD can have high rates of mood and anxiety disorders, and be at high risk for suicide (20-22). They may have increased illness burden, poorer prognosis and delayed response to treatment (23-26). MUD can lead to long-term, persistent, emotional, behavioral and cognitive deficits, increased socio-economical problems and reduced quality of life (27-29). MUD is highly prevalent in patients with HIV (˜15%)(30-35). Over 20 different medications have been tested in randomized controlled trials (RCTs) for treatment of MUD (21). The recent meta-analyses of these studies concluded that most of the medications have not shown statistically significant benefits. Antidepressants (bupropion(36, 37), mirtazapine and sertraline (38) were not effective. Also, anticonvulsants (Topiramate(38), baclofen (39), gabapentin (39, 40)), antipsychotics (aripiprazole (14, 41), opioid antagonists (naltrexone (42)), and medications for SUD (varenicline (43), atomoxetine (44),) provided insufficient evidence of benefit for reducing MUD. No effects were reported in studies of citicoline (45) and ondansetron (46). The PROMETA protocol, consisting of flumazenil, gabapentin and hydroxyzine, was also tested but was not better that placebo (47). Some studies have shown therapeutic potential of anti-METH monoclonal antibodies (AMMA) (48-50) but RCT results are not available. Psychostimulants were widely tested as treatments for MUD (Dexamphetamine (51), methylphenidate (52) and modafinil (52, 53)). There is low-strength evidence that methylphenidate may reduce METH use.

Neuroinflammation and neurodegeneration may be involved in the pathogenesis of MUD and long term METH use can be associated with decreased dopamine neuron (DA) function and neurodegeneration. Magnetic Resonance Imaging (MRI) studies have demonstrated METH-induced abnormalities in striatal structure and function (54, 36, 42, 55, 56). Patients with MUD (48, 57) had larger putamen volumes than controls. fMRI studies also demonstrated functional fronto-striatal circuit deficits in MUD (58, 59) that are associated with cognitive abnormalities (Ref). PET and postmortem brain studies (10) demonstrated microglial activation in patients with MUD (60). METH-activated microglia (61, 62, 12-14, 38-41, 51, 63-66) produce neurotoxins including: cytokines, chemokines, nitric oxide, superoxide radicals and proteases that cause oxidative stress (67-69), BBB disruption (70, 71) and chronic inflammation (1, 72). Dopamine (DA) neurons are highly susceptible to cell death (2-9). PET studies reported chronic reductions in dopamine transporter (DAT) density and DA release in striatum of patients with MUD (73-77).

The term “subject” as used herein refers to any animal. In some instances, the subject is a mammal. The subject can be a human (e.g., a man, a woman, or a child). The methods can be used in any subject who has or is at risk for developing DA-driven addictions, DA-associated disorders, or neurodegenerative diseases associated with the loss of dopamine neurons. The methods can include selection of a human subject who has or had an addiction, disorder, or disease described herein. Methods for identifying or diagnosing such subjects are known in the art. Subject selection can include obtaining a sample from a subject and testing the sample for an indication that the subject is suitable for selection. The subject can be confirmed or identified, for example, by a health care professional, as having had or having a disorder. As used herein the term “sample”, when referring to the material to be tested for the presence of a biological marker using the method of the invention, includes inter alia tissue, whole blood, plasma, serum, urine, sweat, saliva, breath, exosome or exosome-like microvesicles (U.S. Pat. No. 8,901,284), lymph, feces, cerebrospinal fluid, ascites, bronchoalveolar lavage fluid, pleural effusion, seminal fluid, sputum, nipple aspirate, post-operative seroma or wound drainage fluid. The type of sample used may vary depending upon the identity of the biological marker to be tested and the clinical situation in which the method is used. Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The disorders and diseases described herein can be diagnosed based on the levels of dopamine in e.g., bodily fluids such as the cerebral spinal fluid, or in certain areas of the brain, such as the striatum. Dopamine-driven addictions and dopamine-associated disorders can also be diagnosed, for example, based on the Diagnostic and Statistical Manual of Mental Disorders (DSM-5) and/or additional guidelines published by the American Psychiatric Association, as well as based on brain imaging results.

### Administration

In some embodiments, the term “treating” and “treatment” refers to administering to a subject an effective amount of a composition, e.g., a composition comprising a bile acid and a phenylbutyric acid, so that the subject has a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, treatment can be “prophylaxic treatment, where the subject is administered a composition as disclosed herein (e.g., a composition comprising a bile acid and a phenylbutyric acid) to a subject at risk of developing a neurodegenerative disease as disclosed herein. In some embodiments, treatment is “effective” if the progression of a disease is reduced or halted. In some instances, blood, urine or other lab tests can be used for monitoring treatment and recovery.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. It can also refer to a sufficient amount of a clathrin nanoparticle described herein to retard, delay or reduce the risk of progression of a disease or condition, symptoms associated with a disease or condition or otherwise result in an improvement in an accepted characteristic of a disease or condition when administered according to a given treatment protocol. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. In some instances, the DAT targeting agent (e.g., Vanoxerine or an anti-DAT antibody) can be administered at a dose of between about 0.1 mg/kg to about 10 mg/kg (e.g., between about 0.1 mg/kg to about 6 mg/kg, between about 0.5 mg/kg to about 2 mg/kg) per body weight of the subject. In some instances, the DAT targeting agent is administered at about 1 mg/kg.

The terms “administer,” “administering,” or “administration,” as used herein refers to implanting, absorbing, ingesting, injecting, or inhaling, the inventive drug, regardless of form. In some instances, one or more of the compounds disclosed herein can be administered to a subject nasally or intravenously. For example, the methods herein include administration of an effective amount of compound or compound composition to achieve the desired or stated effect. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

The compositions described herein can be used to target dopamine-rich brain regions, such as the striatum. In some instances, administration of the compositions result in enhanced striatal fiber density, improved cognitive and/or motor functions, and/or decrease in addiction-associated hyperlocomotion and neurotoxicity. Administration of the compositions can also result in increased levels of neurotrophins in the striatum, and prevent or reduce elevation of dopamine levels in the subject's brain. In some instances, administration of the compositions can result in reduced frequency of addictive behaviors, or reduced desire for the addictive substance or activities by the subject. In some instances, the methods of treatment increases the number of dopamine secreting neurons in brain regions such as the substantia nigra.

### Pharmaceutical Formulations

A therapeutically effective amount of the compositions described herein (e.g., a composition comprising a clathrin nanoparticle linked to a DAT targeting agent and/or a neurotrophin) can be incorporated into pharmaceutical compositions suitable for administration to a subject, e.g., a human. Such compositions typically include the composition and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions, e.g., an inhibitor of degradation of the ligand.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the composition (e.g., an agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL™ (sodium carboxymethyl starch), or corn starch; a lubricant such as magnesium stearate or STEROTES™; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration. In one aspect, the pharmaceutical compositions can be included as a part of a kit.

Generally the dosage used to administer a pharmaceutical compositions facilitates an intended purpose for prophylaxis and/or treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., Chapter 27 In: “Remington's Pharmaceutical Sciences”, 18th Ed., Gennaro, ed., Mack Publishing Co., Easton, Pa., 1990). Generally, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., Chapter 3, In: Goodman & Gilman's “The Pharmacological Basis of Therapeutics”, 9th Ed., Hardman et al., eds., McGraw-Hill, New York, N.Y., 1996).

### Combination Therapy

The methods of treating a dopamine disorder (e.g. MUD) described herein can further include administering one or more additional therapeutic agent. Exemplary additional agents include aripiprazole, baclofen, bupropion, d-AMP and MPH-SR, dextroamphetamine, gabapentin, ibudilast, methylphenidate, mirtazapine, modafinil, NAC, naltrexone, rivastigmine, and topiramate. Nonpharmacological treatments are also contemplated herein as part of a combination treatment, such as transcranial magnetic stimulation and neurofeedback therapy.

### Kits

The present invention also includes kits, e.g., for use in the methods described herein. In some embodiments the kit comprise one or more doses of a composition described herein. The composition, shape, and type of dosage form for the induction regimen and maintenance regimen may vary depending on a subjects requirements. For example, dosage form may be a parenteral dosage form, an oral dosage form, a delayed or controlled release dosage form, a topical, and a mucosal dosage form, including any combination thereof.

In a particular embodiment, a kit can contain one or more of the following in a package or container: (1) one or more doses of a composition described herein; (2) one or more pharmaceutically acceptable adjuvants or excipients (e.g., a pharmaceutically acceptable salt, solvate, hydrate, stereoisomer, and clathrate); (3) one or more vehicles for administration of the dose; (5) instructions for administration. Embodiments in which two or more, including all, of the components (1)-(5), are found in the same container can also be used.

When a kit is supplied, the different components of the compositions included can be packaged in separate containers and admixed immediately before use. Such packaging of the components separately can permit long term storage without losing the active components' functions. When more than one bioactive agent is included in a particular kit, the bioactive agents may be (1) packaged separately and admixed separately with appropriate (similar of different, but compatible) adjuvants or excipients immediately before use, (2) packaged together and admixed together immediately before use, or (3) packaged separately and admixed together immediately before use. If the chosen compounds will remain stable after admixing, the compounds may be admixed at a time before use other than immediately before use, including, for example, minutes, hours, days, months, years, and at the time of manufacture.

The compositions included in particular kits of the present invention can be supplied in containers of any sort such that the life of the different components are optimally preserved and are not adsorbed or altered by the materials of the container. Suitable materials for these containers may include, for example, glass, organic polymers (e.g., polycarbonate and polystyrene), ceramic, metal (e.g., aluminum), an alloy, or any other material typically employed to hold similar reagents. Exemplary containers may include, without limitation, test tubes, vials, flasks, bottles, syringes, and the like. As stated above, the kits can also be supplied with instructional materials. These instructions may be printed and/or may be supplied, without limitation, as an electronic-readable medium, such as a floppy disc, a CD-ROM, a DVD, a Zip disc, a video cassette, an audiotape, and a flash memory device. Alternatively, instructions may be published on an internet web site or may be distributed to the user as an electronic mail.

### EXAMPLES

The invention is further described in the following examples, which do not limit the scope of the invention described in the claims.

### Example 1. Preparation of Clathrin Cages

To prepare clathrin cages, cDNA encoding residues 1-1074 of the clathrin heavy chain is cloned into the pET23d vector (Novagen) between the NcoI (234) and XhoI (158) sites to generate clathrin fragments containing the terminal domain and the distal domain, and having a C-terminal polyhistidine tag. Next, cDNA corresponding to residues 1074-1675 of the clathrin heavy chain is cloned into vector pET15b (Novagen) between the BamHI (319) and XhoI (324) sites, generating fragments containing the proximal leg domain and the central trimerization domain with an N-terminal polyhistidine tag. The vectors are expressed in E. coli by induction with 0.8 mM isopropyl-B-D-thiogalactopyranoside for 3 hours at 30° C. Expressed proteins are purified from bacterial lysate in binding buffer (50 mM Tris-HCl (pH7.9), 0.5M NaCl, 5 mM imidazole) in a nickel affinity resin using the polyhistidine tag. Proteins are eluted with 100 mM EDTA and dialyzed against 50 mM Tris-HCl (pH7.9). Hub fragments are further purified using size exclusion chromatography on a Superose 6 column (Pharmacia).

The terminal and distal domains and the hub domain are subjected to assembly reactions by overnight dialysis at 4° C. in the assembly buffer (100 mM 2-(N-morpholino) ethanesulfonic acid, pH 6.7, 0.5 mM MgCl₂, 1 mM EGTA, 1 MM Tris (2-carboxyethyl)-phosphine hydrochloride, 3 mM CaCl₂)). Following centrifugation for 5 minutes at 12,000 rpm, the supernatant is then centrifuged for 45 minutes at 45,000 rpm (100,000.times.g). The pellets are resuspended in the assembly buffer, and protein composition is determined by SDS-PAGE. The efficiency of cage formation can be determined by electron microscopy by diluting assembly reactions 1:5 in 10 mM Tris pH7.9, and placing aliquots on a glow-discharged carbon-coated grid, using 1% uranyl acetate as the stain. Cage formation is assessed by counting the numbers of cages having closed, defined edges and visible hexagonal/pentagonal lattice structure.

### Example 2. Production of DAT-Ab-BDNF-Clathrin Nanoparticle (DAT-Ab-BDNF-CNPs)

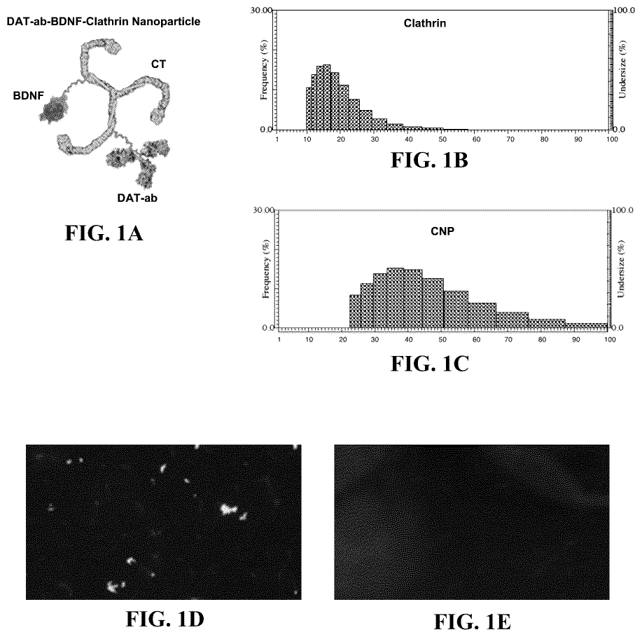

DAT-ab-BDNF-CNPs were synthesized through conjugation of BDNF and DAT antibody (DAT-ab, Santa Cruz-32258) to the heavy chains of clathrin triskelia (CT) via PEGs (FIG. 1A). ELISA demonstrated that on average one molecule of DAT-ab and three molecules of BDNF were attached to CT. The mean hydrodynamic radius (RH) of unconjugated CT was 16.8±5.6 nm, as determined by dynamic light scattering (FIG. 1B). The mean RH increased to 42.13±14.8 nm after BDNF and DAT-ab conjugation to CT via PEGs (FIG. 1C). CNPs with DAT-ab (FIG. 1D) but not with IgG (FIG. 1E) labeled in vitro mouse DAT in striatal slices, confirming specificity of the CNP.

### Example 3. Localization of DAT-Ab-BDNF-CNP to DA Brain Regions after Intranasal (i.n.) Delivery

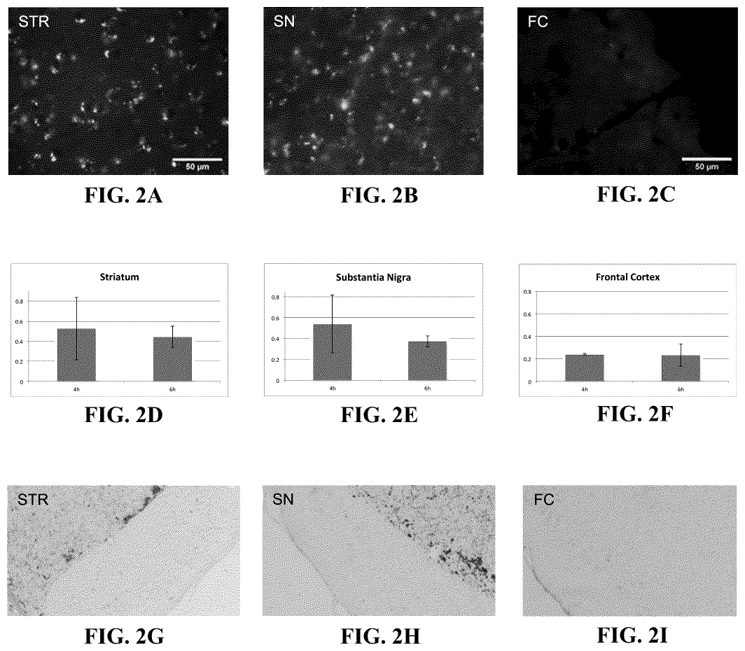

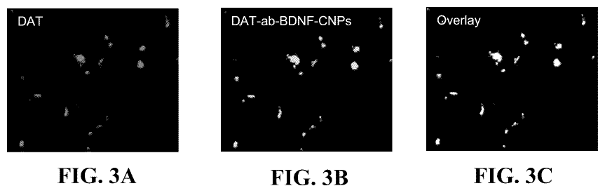

In vivo studies using fluorescent, radioactive, or iron oxide labeled DAT-ab-BDNF-CNPs (0.3 mg/kg of BDNF, 0.55 mg/kg of DAT-ab and 2.4 mg/kg of CT) demonstrate that the i.n. delivered CNPs efficiently bypassed the blood brain barrier (BBB), remained stable, and targeted DAT in DA brain regions in rodent models (FIGS. 2, 3).

To assess localization of the DAT-ab-BDNF-CNPs, brain tissues were collected from mice 4 hours after i.n. delivery of fluorescent NSH-fluorescein labeled DAT-ab-BDNF-CNPs. Fluorescent DAT-ab-BDNF-CNPs were detected in the mouse striatum and substantia nigra, but not the frontal cortex (FIGS. 2A-C).

To further assess localization of these CNPs, DAT-ab-BDNF-CNPs were radiolabeled with ³H-NSP (PerkinElmer, CT). ³H-DAT-ab-BDNF-CNPs were then dialyzed in PBS buffer (pH=7.4) to remove free labels by using Biotech CE membranes (100 kDa, Spectrum Labs) until the counts per minute (CPM) in the last three buffer solutions were equal to the background level. Inducible HIV-1 Tat transgenic (iTat) mice received i.n. administration of 9 μCi of ³H-DAT-ab-BDNF-CNPs, and brains were removed 4, 6 and 24 hours after administration. Discrete brain areas were micro-dissected, weighed, homogenized and dissolved in Solvable per PerkinElmer's instructions (6NE9100). Hionic-Fluor (PerkinElmer) scintillation cocktail (4 mL) was added to each vial, and ³H-DAT-ab-BDNF-CNPs concentrations in brain regions, including the striatum, substantia nigra, and frontal cortex, were determined using scintillation counting methods. The highest concentration of DAT-ab-BDNF-CNPs in DA brain regions was detected after 4 h (FIGS. 2D-2F).

In addition, iron-labeled MRI DAT-ab-BDNF-CNPs were i.n. delivered to F344 rats and detected with iron staining in the rat striatum and substantia nigra, but not in the prefrontal cortex, 4 hours after delivery (FIGS. 2G-2I).

To confirm that the DAT-ab-BDNF-CNPs target DAT specifically, iTat mice were treated with a single i.n. dose of fluorescent NSH-fluorescein labeled DAT-ab-BDNF-CNPs, and brains were collected 4 hours after treatment. The anti-DAT antibody was detected with Rhodamine-conjugated anti-rat secondary antibody, and robust immunoreactivity was observed in the striatum (FIG. 3A). NSH-Fluorescein labeled DAT-ab-BDNF-CNPs were also observed abundantly throughout the striatum following i.n. delivery (FIG. 3B). Confocal fluorescent microscopy revealed regions positive for both DAT and NSH-fluorescein labeled DAT-ab-BDNF-CNPs throughout the striatum. Clear overlapping of the two labels (yellow) was detected (FIG. 3C) indicating that intact and functional DAT-ab-BDNF-CNPs targeted and colocalized with DAT in vivo.

### Example 4. DAT-Ab-BDNF-CNPs for Treatment of Methamphetamine (METH) and/or Tat-Induced Neurotoxicity

The ability of DAT-ab-BDNF-CNPs to rescue neurotoxicity was assessed using one, or a combination, of the following two mouse models: 1) mice receiving methamphetamine, and/or 2) doxycycline-inducible HIV-neuroAIDs/Tat transgenic mouse model (iTat).

iTat mice were randomly assigned to 8 different groups (n=4-5/group) listed in Table 1.

TABLE 1

| | i.p. dox (Tat+) | i.p. METH | i.n. DAT-ab-BDNF- |
|---|---|---|---|
| | or saline (Tat−) | or N/A | CNPs or Sal |
| 1 | Tat− | N/A | Sal |
| 2 | Tat+ | N/A | Sal |
| 3 | Tat− | METH | Sal |
| 4 | Tat+ | METH | Sal |
| 5 | Tat− | N/A | DAT-ab-BDNF-CNPs |
| 6 | Tat+ | N/A | DAT-ab-BDNF-CNPs |
| 7 | Tat− | METH | DAT-ab-BDNF-CNPs |
| 8 | Tat+ | METH | DAT-ab-BDNF-CNPs |

Saline control groups (1-4) received i.n. saline (Sal) daily for 7 days. Study groups (5-8) received i.n. DAT-ab-BDNF-CNPs (0.3 mg/kg of BDNF, 0.55 mg/kg of DAT-ab and 2.4 mg/kg of CT) daily for 7 days. Mice also received intra-peritoneal (i.p.) injections of doxycycline (Dox, 100 mg/kg; Tat+) or saline (Tat−) daily for 7 days. On the 6th day, specific groups of mice (denoted with METH in Table 1) also received methamphetamine hydrochloride (D-METH HCl; 4 mg/kg; Sigma) i.p. at 11 AM and then every 2 hours, for a total of 4 injections. On the 8th day mice were anesthetized, perfused with PBS transcardially, and decapitated, and half the brain was immediately frozen, while the other half was drop-fixed in 4% paraformaldehyde for 48 hours. Brains were cryoprotected in 30% sucrose, and cut in 20 μm coronal sections on a cryotome.

The ability of DAT-ab-BDNF-CNPs to reverse METH and/or HIV/Tat-induced toxicity in the striatum was assessed using immunohistochemistry (IHC) for tyrosine hydrolase (TH) positive fibers in the striatum. Coronal sections were permeabilized, blocked, and incubated in anti-TH antibody (1:100, Santa Cruz, SC-25269) for 24 hours at 4° C. Coronal sections were then incubated with anti-mouse Alexa Fluor 568 secondary antibody (1:200, Fisher) for 2 hours at room temperature. Slides were cover-slipped, and sections containing the striatum were matched and imaged at 20× (air) with Hammamatsu ORCA ER CCD camera on a Zeiss Axio A1 microscope (Carl Zeiss Inc.) using Micro-Manager (NIH). The square region of interest (ROI) was defined within the striatal region, and the mean pixel intensity was computed with Image J (NIH) for each ROI from every 6th section. The imaging threshold was held constant for the analysis of a series of sections. An investigator was blinded to animal treatment status.

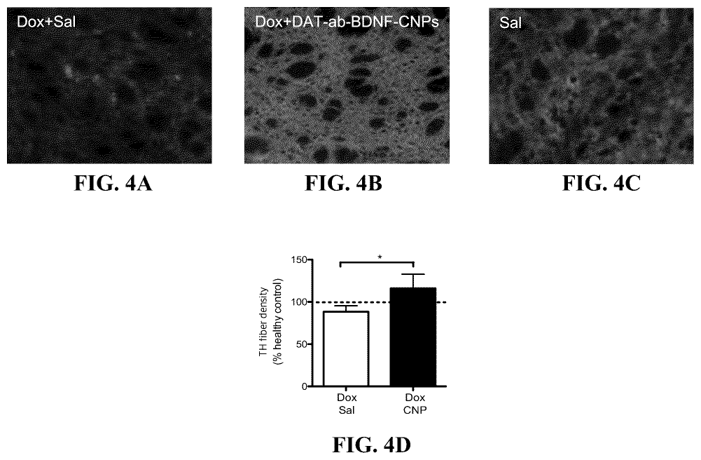

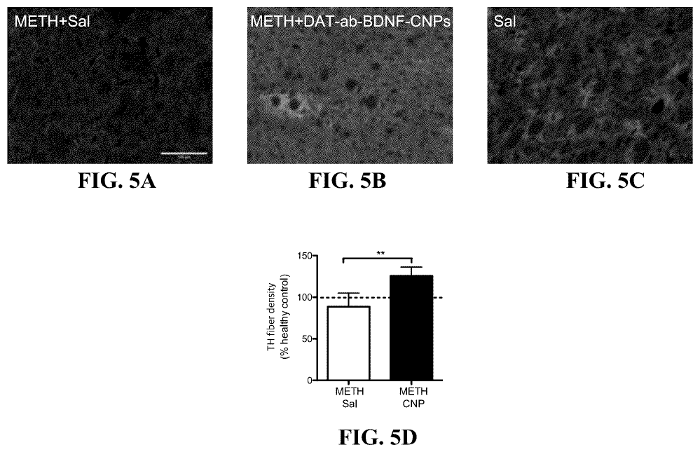

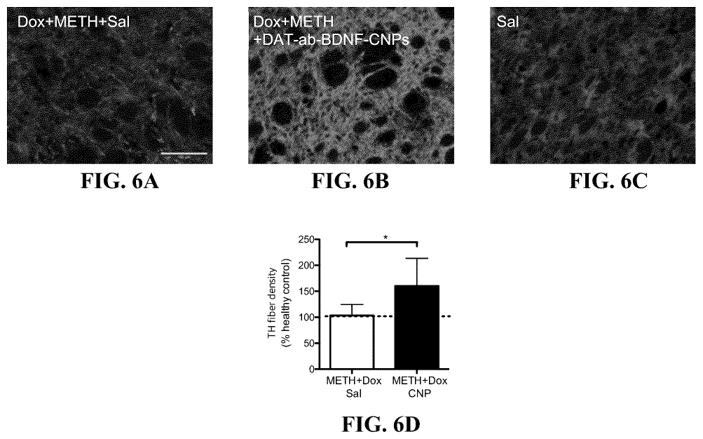

DAT-ab-BDNF-CNPs reversed Tat neurotoxicity and improved density of TH+ fibers in the striatum. TH immunoreactivity was significantly higher (p-value=0.0284) in the striatum of Tat+ mice treated with DAT-ab-BDNF-CNPs compared to saline-treated Tat+ controls (FIGS. 4A-4D). DAT-ab-BDNF-CNPs also reversed METH toxicity, as TH immunoreactivity was significantly higher (p-value=0.0022) in the striatum of Tat− mice exposed to METH and treated with DAT-ab-BDNF-CNPs vs. saline (FIGS. 5A-D). Furthermore, DAT-ab-BDNF-CNPs reversed the combined METH and Tat toxicity in mice as TH immunoreactivity was significantly higher (p-value=0.047) in the striatum of Tat+ mice exposed to METH and treated with DAT-ab-BDNF-CNPs vs. saline (FIGS. 6A-D).

These results demonstrate that intranasally delivered DAT-ab-BNDF-CNPs efficiently bypassed the BBB, targeted DA brain regions, and delivered 60 ng/ml of BDNF to the mouse striatum, which is over 100 fold higher than striatal BDNF concentrations reported in previous BDNF systemic delivery studies (Giampa et al. PLoS One 8(5): p. e64037, 2013). The DAT-ab-BDNF-CNPs also reversed HIV/Tat and/or METH-induced neurotoxicity by improving density of TH positive fibers in the striatum of mice.

### Example 5: Preparation and Characterization of GBR-BDNF-CNPs and GBR-CNPs

GBR-BDNF-CNPs were prepared by crosslinking the GBR12909 drug and BDNF to CNPs via PEGs. Clathrin triskelia were isolated from fresh pig brains according to standard procedures. The clathrin sequence is very well preserved between species. Protein concentrations were determined by Bradford protein assay. The chemical reactivity of cysteines of clathrin triskelia were used to attach PEGs, which were then used to crosslink one BDNF and four GBR molecules to each clathrin heavy chain (MW=180 kDa) for optimal delivery. PEGs stabilized BDNF molecules, prevented degradation of BDNF by enzymes, and extended the half-life of BDNF for days.

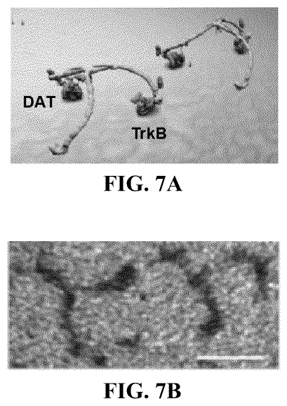

GBR-BDNF-CNP purity is analyzed by SDS-PAGE using Coomassie-blue staining. Analysis of CNP size and structure is performed on a Joel 1200EX transmission electron microscope (TEM) (FIG. 7A-B). For quality control, automated characterization of CNP size is performed with Dynamic Light Scattering (DSL) (S90, Brookhaven Instruments, CT). CNPs with diameters of less than 60 nm are less likely to be picked up by the reticuloendothelial system (RES).

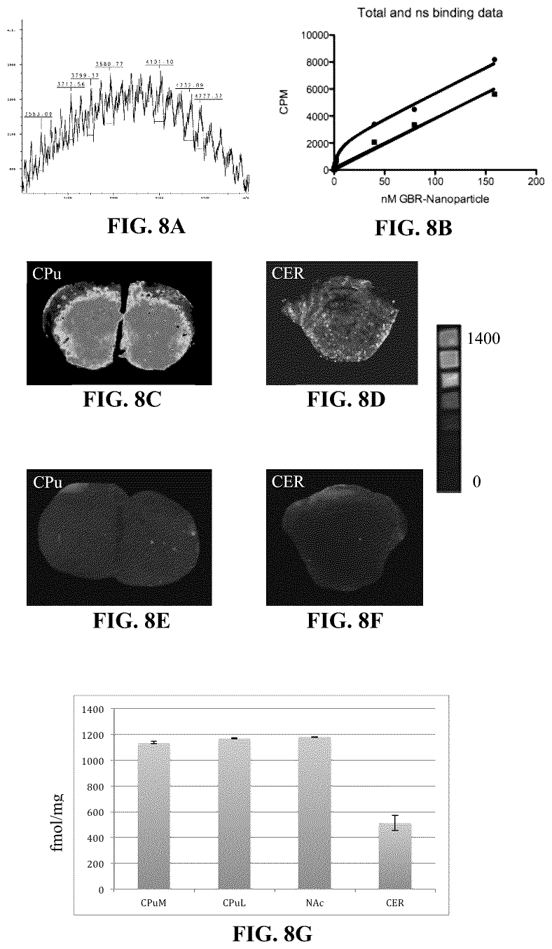

CNPs crosslinked with GBR bound to dopamine brain regions both in vitro and in vivo. MALDI-TOF analysis indicated that the molecular weight of the PEGylated GBR ligands was 4 kDa (FIG. 8A). To demonstrate binding specificity in vitro, PEGylated GBR ligands were cross-linked to ¹⁴C radiolabeled clathrin heavy chains (4:1). Rat striatal slices were incubated for 120 minutes with 10 different concentrations of ¹⁴C-GBR-CNPs (0.06 to 158.7 nM) in 100 μL of PBS (pH 7.4). For nonspecific binding of CNP, 1 μM of cold GBR was used. Kd value was 3 (FIG. 8B). Analyses of autoradiograms demonstrated binding of ¹⁴C-GBR-clathrin to rat brain regions, including the caudate-putamen (CPu) (FIGS. 8C-8F). CNP specific binding (total binding-nonspecific binding) were analyzed in the CPu, nucleus accumbens and cerebellum (FIG. 8G). The striatum-to-cerebellum ratio of CNP specific-binding was 2.3.

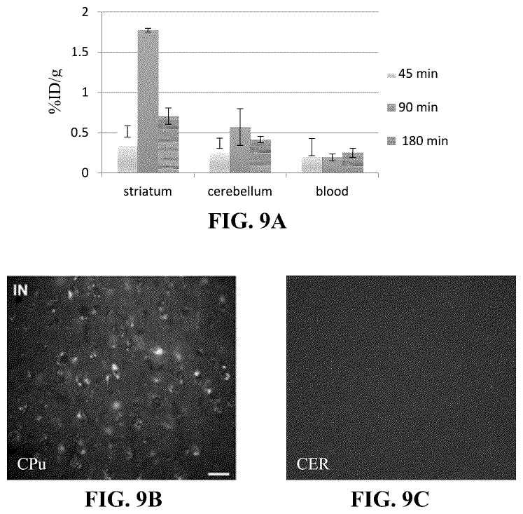

In vivo studies confirmed specific targeting of DAT with GBR-CNPs. Rats were sacrificed in groups of 4 at 45 minutes, 90 minutes and 180 minutes post intravenous injection of radioactive ¹⁵³GdDTPA-GBR-CNPs. Mean percentages of the injected dose per gram of tissue (% ID/g) in brain regions and blood were determined. The highest concentration (1.77% ID/g) of CNPs was observed in the rat striatum 90 minutes after i.v. delivery. There were 2.5 times more GBR-CNPs in the striatum compared to cerebellum (0.70% ID/g) (FIG. 9A).

Fluorescent FITC-GBR-BDNF-CNPs were delivered i.n. to rats. FITC-GBR-BDNF-CNPs were detected in the rat CPu but not the CER 90 minutes after delivery of these CNPs. As shown in FIG. 9B-9C, CNPs with both GBR and BDNF attached (38 nm in size) bypass the BBB, target dopamine brain regions, and deliver BDNF to targeted DAT-rich brain regions.

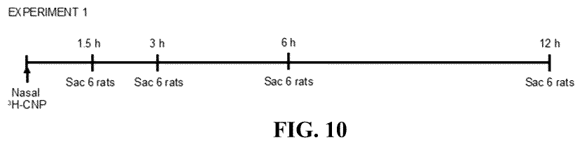

Rats treated intranasally with GBR-BDNF-CNPs are used to determine both the in vivo biodistribution of GBR-BDNF-CNPs as well as the time course (pharmacokinetics) for accumulation and clearance of GBR-BDNF-CNPs from rat brain and organs (FIG. 10). A total of 24 (12 males and 12 females) Fisher 344 rats are administered with ³H-GBR-BDNF-CNPs (6 animals for each of the four time points). Rats are placed in a supine position, and a volume of 100 μL of PBS treatment solution is delivered in nose drops (10 μL per drop) over a period of 20 minutes, alternating between each naris every 1.5 minutes. Animals are sacrificed and GBR-BDNF-CNP distribution is evaluated at 4 time points (1.5 hours, 3 hours, 6 hours, and 12 hours) after administration. At the end of each time point, blood samples, superficial and deep lymph nodes, nasal turbinates, and organs, including the brain, lung, heart, liver, spleen, pancreas, stomach, intestine, and kidney, are removed. Olfactory bulbs and discrete brain areas are micro-dissected. Tissues are weighed, homogenized, and dissolved in Solvable according to manufacturer instructions (PerkinElmer #6NE9100).

To compare and determine a time course for accumulation and clearance of GBR-BDNF-CNPs from the rat brain and organs, ³H-GBR-BDNF-CNP concentrations are determined in the blood and organ samples by liquid scintillation counting methods. This analysis provides information on the time point with the greatest degree of accumulation of GBR-BDNF-CNPs in brain regions with DAT (e.g. olfactory bulb, striatum, substantia nigra, etc.) The maximum plasma and brain tissue CNP concentrations (Cₘₐₓ) are determined, as well as the corresponding time (tₘₐₓ). The area under the plasma or organ concentration-time curve (AUC_{0-360 min}) are calculated using GraphPad Prism 8 (GraphPad Software, US). All AUC values and AUC_{brain}/AUCₚₗₐₛₘₐ ratios are calculated per individual animal before determining the mean value. Multiplying the individual AUC values by total blood volume, or weight of tissue, gives the cumulative amount of drug delivered to the blood or organ tissue. The percentages of injected dose per gram of wet tissue (% ID/g) are also determined.

Rats are acquired from Charles River and housed individually at McLean Hospital Animal Facility using a 12-hour light-dark cycle (lights on at 07:00), with food and water provided ad libitum. Rats are treated in accordance with all relevant institutional, state, and federal guidelines.

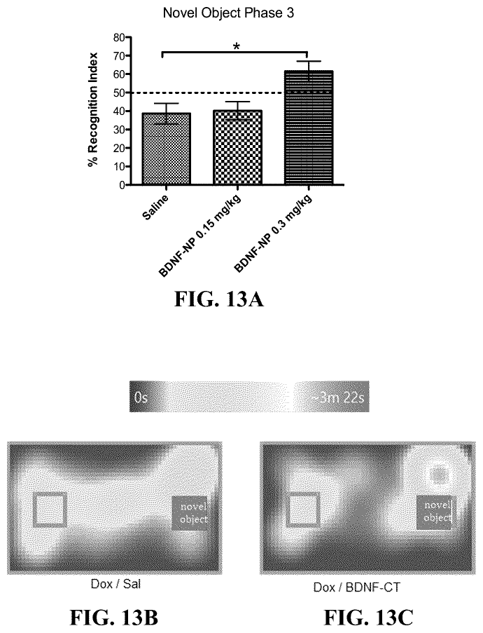

Rats are dosed with 300 μg/kg of BDNF. This BDNF dosage was shown to be effective in cognitive studies (FIGS. 13A-C) with escalating doses of BDNF-CNPs, and in striatal IHC studies of TH+ fiber densities (FIGS. 4-6).

Rats are dosed with 1 mg/kg of GBR12909 via nasal administration that decreases systemic exposure. This GBR12909 dose of 1 mg/kg is five times lower than the GBR12909 dose used in a Phase I study on vanoxerine for treatment of atrial fibrillation/atrial flutter (which used 5 mg/kg).

### Example 6. GBR-BDNF-CNPs for Treatment of Cognitive and Motor Deficits in Rats Exposed to Toxic Binge Doses of METH

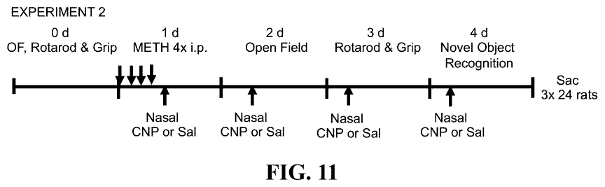

FIG. 11 illustrates the setup for assessing the effect of GBR-BDNF-CNP treatment on rats exposed to toxic binge doses of METH. The ability of CNPs to reverse METH-induced neurotoxicity and improve cognitive and motor functions in rats are assessed using behavioral tests and ex vivo assays.

### 1. Behavioral Tests

On the 1st day, F344 rats (n=48, 24 males and 24 females) (250-300 g) are randomized in two study groups (i.n. GBR-BDNF-CNPs or saline control). Age/sex matched F344 rats (n=24) are used as healthy saline controls. Both study groups receive i.p. D-METH HCl (10 mg/kg; Sigma) at 8:00 AM and then every 2 hours, for a total of 4 injections. Body temperatures are measured rectally 30 minutes prior to METH administration and then measured every 90 min for up to 8 hours. Animals with a body temperature of 41.5° C. or greater are immediately placed over crushed ice until the body temperature is reduced to at least 40° C. This binge dose regimen has been used in animal studies of METH addiction and is shown to induce microglial activation, synaptic damage and neurodegeneration in rodents. After the last METH dose, rats receive i.n. GBR-BDNF-CNPs (300 μg/kg of BDNF and 1 mg/kg of GBR) or Sal daily for 4 days. On the 4th day, after completing all cognitive and motor tests, rats are euthanized, brains will be removed, post-fixed and cryoprotected.

Motor and memory functions are assessed by blinded observers using the following tests: Open Field (OF), rotarod performance, forelimb grip strength, and Novel Object Recognition (NOR) tests. These tests have been successfully used in different animal models to evaluate balance, coordination, strength and memory, are automated and have translational research values.

The Open Field test (OF) is administered to evaluate spontaneous locomotor activity, exploration, and rearing activity [52]. On day 2, rats are placed in the lower left corner of a square Plexiglas box (Any-Box system; Stoelting, IL) and allowed to explore for 20 min. Movements are monitored, recorded and digitally encoded by AnyMaze (6.0) image capture software with Any-Box system (Stoelting). The average speed and total distance traveled, time spent mobile, and time spent rearing are used as indices of motor/exploratory behavior.

The rotarod performance test (TNE, US) is used to assess CNP effects on balance and motor coordination in METH-treated rats. First, rats are trained to balance on a rotarod (3 cm in diameter and suspended 44.5 cm high; at 5-10 rpm) for 30 seconds. Rats are then tested on three accelerating speed trials (180 seconds maximum latency at 0-30 rpm) on days 0 (baseline) and 3. The mean latency to fall from the rotarod across the three accelerated trials is utilized as an index for locomotor performance.

Forelimb grip strength test (Chatillon, DFE II N10, Ametek, Largo, FL) is used to assess CNP effects on motor strength in METH-treated rats. Rats are lowered toward the instrument until the bar is gripped firmly with forelimbs. The rat is then gently pulled away from the bar. Peak force is registered in grams-force. Each rat is tested 5 times on days 0 (baseline) and 3. Grip strength is normalized by body weight (g). Treatment with GBR-BDNF-CNPs is expected to enhance motor strength.

Novel Object Recognition (NOR) Test is a simple learning test consisting of three 10 minute phases, separated by 10-min inter-trial intervals. It has been demonstrated that METH-treated rats exhibited deficits on this test. Rats are tested on day 4. During all phases, time spent exploring the objects is recorded using the ANY-maze system (Stoelting, IL) and direct observation. Object A does not change during any phase of the task, whereas Object B changes position in Phase II, and is replaced by a novel object in Phase III. For each phase the percent time spent exploring Object B is calculated as the time spent exploring Object B divided by the time spent exploring both objects. Phase III is used to assess memory for the familiar versus novel object. It is expected that GBR-BDNF-CNP vs saline-treated rats will spend more time exploring the novel object in Phase III. This increase in time spent exploring the novel object requires intact memory of the familiar object from the previous phases.

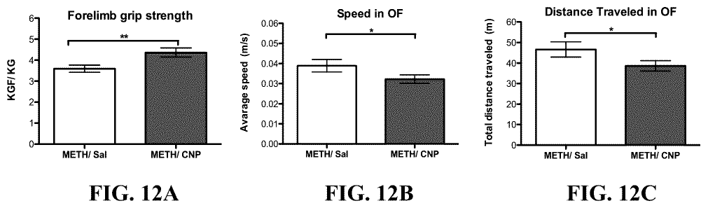

In a preliminary experiment, mice received i.p. saline and i.n. treatments (DAT-ab-BDNF-CNPs or Sal) daily for 7 days. On the 6^{th} day, mice also received i.p. D-METH HCl (4 mg/kg) every 2 hours for a total of four injections. OF and forelimb grip tests were performed on the 7^{th} day. All mice were sacrificed on the 8^{th} day. CNP-treated METH+ mice exhibited higher grip strength, decreased average speed, and decreased distance traveled compared to saline-treated METH+ mice (FIGS. 12A-12C).

Next, iTat mice were induced with 100 mg/kg Dox for 7 days. Concurrently, mice received i.n. either saline; BDNF-CNPs with 0.3 mg/kg of BDNF and 2.4 mg/kg of CTs; or BDNF-CNPs with 0.15 mg/kg of BDNF and 1.2 mg/kg of CTs. Tat+ mice were tested for novel object recognition after the last dose of saline or NPs. Saline treated Tat+ mice spent less time on the novel object (FIGS. 13A, B) than Tat+ mice that received high BDNF-CNP doses (FIGS. 13A, C). Tat+ mice treated with low BDNF-CNP doses were not different from placebo treated Tat+ mice.

For data collected from all the aforementioned motor and memory tests, data is analyzed and presented as the mean±SEM (FIG. 12, FIG. 13A). All the repeated measures are analyzed using Mixed Model (JMP, SAS) with time or trial as the within-subject repeated measure, and treatment group as the between-subject variable. Data from NOR are analyzed using one-way analysis of variance (ANOVA).

### 2. Ex Vivo Analysis

Rats are euthanized by excess carbon dioxide inhalation and decapitation. Upon death, a final blood collection is obtained by cardiac puncture. The brain is removed and divided in half: one hemibrain is snap-frozen in liquid nitrogen and stored at −80° C. for biochemical studies. The other hemibrain is drop-fixed in 4% PFA in PBS for 24 hours at room temperature, sucrose protected in 10-30% sucrose at 4° C. and embedded in OCT (TissueTek) for cryosectioning. 10 μm coronal cryosections are cut and stained.

IHC is performed using ELITE ABC method of Vector Laboratories (Burlingame, CA) and DAB as the chromagen. Double immunofluorescence is performed by blocking in 2% serum, mixing 2 primary antibodies (mAb and pAb or different Ig isotypes) and applying to sections overnight at 4° C., rinsing in 0.1 M Tris, blocking in 2% serum in Tris, mixing two fluorescently-labeled secondary antibodies and applying to sections for 2 hours at RT, rinsing twice in Tris, incubating the sections in 0.3% Sudan Black B in 70% ethanol in the dark for 10 minutes, washing in TBS, washing in water, fixing in formalin for 1 hours in the dark, washing in water, and coverslipping the slides with Hydromount (National Diagnostics, Atlanta). Negative controls for IHC and IF include omission of primary antibody or using mouse IgG as a primary antibody. The primary Abs being used are as follows: Pre-synaptic marker synaptophysin (SYN, D-4, sc-17750); activated pTrkB marker (Y490, Cell Signaling, 9141S); apoptosis marker cleaved caspase-3 (Asp175, 9661S, Cell Signaling); DAT marker (6-5G10, sc32258) and TH marker (TH, F-11, sc-25269) from Santa Cruz Biotech; astroglial marker glial fibrillary acidic protein (GFAP, Z0334, Dako); microglial marker ionized calcium binding adaptor molecule 1 (Iba-1, NCNP24, Wako); and mouse Ig (goat anti-mouse IgG as a negative control). To analyze the image, computer-assisted quantification of synaptic and cell markers is performed using BIOQUANT Image Analysis (Nashville, TN). Six images of immunolabeled sections is captured at equidistant levels (˜100 μm apart) through each rat brain using a Ziess M1 microscope with a motorized stage and a Q-Imaging camera. All images for one experiment is captured on a constant threshold and % area immunoreactivity above threshold calculated.

Levels of BDNF in brain regions (CPu, nucleus accumbens core and shell) of rats treated with GBR-BDNF-CNPs or saline are measured using ELISA. Snap-frozen brain tissues are homogenized in 200 μl of lysis buffer (Promega, Madison, WI). The samples are centrifuged at 14,000 g for 30 min at 4° C., diluted (1:5) in Dulbecco's phosphate-buffered saline (DPBS) with HCl (pH 2.6), and neutralized with NaOH (pH 7.6). The levels of mature BDNF are measured using the BDNF Emax Immuno Assay System (Promega) according to the manufacturer's protocol.

DA and its metabolites are also measured in rats treated with GBR-BDNF-CNPs or saline. The concentrations of DA, 4-dihydroxy-phenylacetic acid (DOPAC), homovanillic acid (HVA) are quantified in striatal tissues by HPLC. Striatal tissues are homogenized in 0.1 M perchloric acid, centrifuged at 10,000 g for 10 min at 4° C. The supernatants are collected, filtered and subjected to HPLC (HTEC-500; Eicom, Kyoto, Japan) with a column (EICOMPAK SC-30DS; Eicom) and detected by an electrochemical detector (AD Instruments, Castle Hill, Australia). The mobile phase consists of 0.1 mM citric acid, 0.1 M sodium acetate, 220 mg/liter octane sulfate sodium, 5 mg/liter EDTA, and 20% methanol, (pH 3.5). DA, DOPAC, and HVA levels are not expected to differ between rats treated with CNPs and healthy controls.

For all ex vivo analyses, group means are statistically compared with ANOVA. The IHC sample size (n=12 per group) provides power (1-0)=0.9 to detect an α=0.05 difference.

### Example 7. Effects of Nasally Delivered GBR-BDNF-CNP on Context-, Cue-, or METH-Induced METH Seeking Behavior in Rats

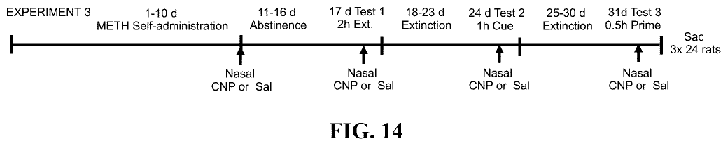

FIG. 14 illustrates the setup for assessing the effects of GBR-BDNF-CNP treatment on METH-seeking behavior in rats. Rats self-administer METH for 10 days. Following a period of abstinence, relapse to METH-seeking behavior elicited by discrete light and tone cues will be assessed. Rats then undergo 6 days of extinction training, followed by an assessment for reinstatement of METH-seeking behavior elicited by light and tone cues. Rats then undergo an additional 6 days of extinction training, followed by an assessment for reinstatement of METH-seeking behavior elicited by a METH-priming injection. GBR-BDNF-CNPs are delivered intranasally and non-invasively following the final METH self-administration session and before reinstatement sessions. The nucleus accumbens core regulates seeking behaviors and BDNF can attenuate the reinstatement to cocaine-seeking behavior by normalizing glutamate activity in the NAc core. It is expected that more BDNF will be delivered to the NAc core than the shell because there is significantly more DAT in the NAc core versus the shell.

F344 rats (n=48, 24 males, 24 females) (250-300 g) are randomized into two treatment groups (GBR-BDNF-CNPs or saline). Age/sex matched F344 rats (n=24) are used as yoked saline controls. A fixed-ratio 1 schedule of food reinforcement (45 mg pellets; Noyes, Lancaster, NH) are used to train rats to lever press. Training is performed in a sound-attenuated operant conditioning rat chamber (Medical Associates, St Albans, VT) during a 16 hour overnight training session. Each chamber is equipped with following items: 2 retractable levers; a white stimulus light above each lever; a food pellet dispenser between the levers; a red house light on the wall opposite the levers; and a speaker connected to a tone generator (Med. Associates). During the session, each lever press on the active (right) lever results in delivery of one food pellet only. Lever presses on the inactive (left) lever has no programmed consequences.

At 48 hours after food training, rats are anesthetized using a mixture of ketamine hydrochloride (66 mg/kg i.p.; Fort Dodge, IA) and xylazine (1.33 mg/kg i.p.; Bayer, KS) followed by Equithesin (0.5 mL/kg). Catheters (0.64 mm i.d., 1.19 mm o.d.; Dow Corning, MI) are implanted into the right jugular vein of rats. The i.v. catheters are then attached to cannulas that are secured to harnesses (Harvard Apparatus, MA). Following surgery, rats are infused i.v. with 0.1 mL each of cefazolin (100 mg/mL) and heparinized saline (70 U/mL) once daily during a 5-day recovery period. Catheter patency is verified by infusing 0.1 mL of methohexital sodium (20 mg/mL i.v., Eli Lilly, IL), which induces a rapid loss of muscle tone.

### 1. METH Self-Administration

METH self-administration (SA) is performed in operant conditioning rat chamber (Medical Associates). The infusion line is tethered to a liquid swivel (Instech, PA). Rats receive 0.1 mL of heparinized saline (10 U/mL, i.v.) prior to each SA session. Animals are then connected to the infusion tether. The red house light signals the beginning of a session. METH (MiliporeSigma-, St. Louis, MO) is delivered using a computer-controlled infusion pump. A computerized data collection program (MED PC) is used to control the entire system. A fixed ratio 1 schedule of METH reinforcement is paired with a conditional stimulus (CS). The CS consists of 2 components: activation of a white light above the active lever and generation of tone (4.5 kHz, 78 dB). Responses on the active lever results in delivery of METH (0.02 mg/0.05 mL bolus) over 2 seconds. Each infusion is followed by a 20 second ‘time-out’ period when active lever presses is recorded, but does have no programmed consequences. Responses on the inactive (left) lever is also recorded. The maintenance criterion that rats have to reach for METH SA is at least 10 infusions of METH per 2 hour session for 10 days. Yoked saline controls receive 50 μl infusions of 0.9% sterile saline whenever the matched self-administering subject receives a METH infusion. Rats self-administer METH on 10 consecutive days during 2 hour daily sessions.

### 2. Abstinence, Extinction, and Reinstatement in Rats

After the final (10th) SA session, rats receive i.n. GBR-BDNF-CNPs or saline. Rats remain in the home cage for 6 days following the last SA session. On day 7 of abstinence, rats receive i.n. CNP or Sal at 9 AM and take a 2 hour test in the self-administration chambers at 11 AM. Each active lever press results in a contingent 5 second presentation of light and tone CS (with a 20 second time-out) in the absence of METH reinforcement. Rats then have extinction sessions daily for 6 days. Lever presses have no programmed consequences during these sessions. To meet extinction criteria, responses on the active lever need to be <25 presses for 2 consecutive days. On day 14 of abstinence, rats receive i.n. GBR-BDNF-CNPs or Sal at 9 AM. At 11 AM, rats have a 1 hour extinction session followed immediately by a 1 hour cue-induced reinstatement test. During the cue reinstatement test, active lever presses result in light+ tone CS presentations in the absence of METH reinforcement. Rats then have 6 additional days of extinction. On day 21 of abstinence, rats receive i.n. GBR-BDNF-CNPs or Sal at 9 AM. At 11 AM, rats undergo a 1 hour extinction session followed by a 30 minute METH-primed reinstatement test. Before this test, rats are injected with METH (1 mg/kg, i.p.). During the METH-primed reinstatement test, lever presses are recorded, but have no programmed consequences. At the end of this final test, rats are euthanized and the brains are harvested for IHC evaluations and BDNF and DA analysis (exemplary assays and analyses are described in Example 7). Behavioral data are analyzed with a two-way mixed-factor ANOVA. The sample size (n=24 per group) can provide power (1-(3)=0.8 to detect an α=0.05 difference.

### REFERENCES

1. Chang, L., et al., Structural and metabolic brain changes in the striatum associated with methamphetamine abuse. Addiction, 2007. 102 Suppl 1: p. 16-32.
2. Riddle, E. L., A. E. Fleckenstein, and G. R. Hanson, Mechanisms of methamphetamine-induced dopaminergic neurotoxicity. AAPS J, 2006. 8(2): p. E413-8.
3. Krasnova, I. N. and J. L. Cadet, Methamphetamine toxicity and messengers of death. Brain Res Rev, 2009. 60(2): p. 379-407.
4. Cadet, J. L. and I. N. Krasnova, Molecular bases of methamphetamine-induced neurodegeneration. Int Rev Neurobiol, 2009. 88: p. 101-19.
5. Thrash, B., et al., Methamphetamine-induced neurotoxicity: the road to Parkinson's disease. Pharmacol Rep, 2009. 61(6): p. 966-77.
6. Kiyatkin, E. A. and H. S. Sharma, Environmental conditions modulate neurotoxic effects of psychomotor stimulant drugs of abuse. Int Rev Neurobiol, 2012. 102: p. 147-71.
7. Krasnova, I. N., Z. Justinova, and J. L. Cadet, Methamphetamine addiction: involvement of CREB and neuroinflammatory signaling pathways. Psychopharmacology (Berl), 2016. 233(10): p. 1945-62.
8. Ersche, K. D., et al., Meta-analysis of structural brain abnormalities associated with stimulant drug dependence and neuroimaging of addiction vulnerability and resilience. Curr Opin Neurobiol, 2013. 23(4): p. 615-24.
9. Berman, S., et al., Abuse of amphetamines and structural abnormalities in the brain. Ann N Y Acad Sci, 2008. 1141: p. 195-220.
10. Baicy, K. and E. D. London, Corticolimbic dysregulation and chronic methamphetamine abuse. Addiction, 2007. 102 Suppl 1: p. 5-15.
11. Jernigan, T. L., et al., Effects of methamphetamine dependence and HIV infection on cerebral morphology. Am J Psychiatry, 2005. 162(8): p. 1461-72.
12. Sabrini, S., et al., Methamphetamine use and cognitive function: A systematic review of neuroimaging research. Drug Alcohol Depend, 2019. 194: p. 75-87.
13. Droutman, V., et al., Neurocognitive decision-making processes of casual methamphetamine users. Neuroimage Clin, 2019. 21: p. 101643.
14. Hart, C. L., et al., Is cognitive functioning impaired in methamphetamine users? A critical review. Neuropsychopharmacology, 2012. 37(3): p. 586-608.
15. Volkow, N. D., et al., Loss of dopamine transporters in methamphetamine abusers recovers with protracted abstinence. J Neurosci, 2001. 21(23): p. 9414-8.
16. Volkow, N. D., et al., Association of dopamine transporter reduction with psychomotor impairment in methamphetamine abusers. Am J Psychiatry, 2001. 158(3): p. 377-82.
17. Sekine, Y., et al., Association of dopamine transporter loss in the orbitofrontal and dorsolateral prefrontal cortices with methamphetamine-related psychiatric symptoms.

Am J Psychiatry, 2003. 160(9): p. 1699-701.
18. Sekine, Y., et al., Methamphetamine-related psychiatric symptoms and reduced brain dopamine transporters studied with PET. Am J Psychiatry, 2001. 158(8): p. 1206-14.
19. McCann, U. D., et al., Persistent cognitive and dopamine transporter deficits in abstinent methamphetamine users. Synapse, 2008. 62(2): p. 91-100.
20. Doyle, K., et al., Aging, prospective memory, and health-related quality of life in HIV infection. AIDS Behav, 2012. 16(8): p. 2309-18.
21. Chan, B., et al., Pharmacotherapy for methamphetamine/amphetamine use disorder—a systematic review and meta-analysis. Addiction, 2019. 114(12): p. 2122-2136.
22. Lee, N. K., et al., Pharmacotherapy for amphetamine dependence: A systematic review. Drug Alcohol Depend, 2018. 191: p. 309-337.
23. Bhatt, M., et al., Efficacy and safety of psychostimulants for amphetamine and methamphetamine use disorders: a systematic review and meta-analysis. Syst Rev, 2016. 5(1): p. 189.
24. Elkashef, A. M., et al., Bupropion for the treatment of methamphetamine dependence. Neuropsychopharmacology, 2008. 33(5): p. 1162-70.
25. Colfax, G. N., et al., Mirtazapine to reduce methamphetamine use: a randomized controlled trial. Arch Gen Psychiatry, 2011. 68(11): p. 1168-75.
26. Shoptaw, S., et al., Randomized, placebo-controlled trial of sertraline and contingency management for the treatment of methamphetamine dependence. Drug Alcohol Depend, 2006. 85(1): p. 12-8.
27. Elkashef, A., et al., Topiramate for the treatment of methamphetamine addiction: a multi-center placebo-controlled trial. Addiction, 2012. 107(7): p. 1297-306.
28. Heinzerling, K. G., et al., Randomized, placebo-controlled trial of baclofen and gabapentin for the treatment of methamphetamine dependence. Drug Alcohol Depend, 2006. 85(3): p. 177-84
29. Coffin, P. O., et al., Aripiprazole for the treatment of methamphetamine dependence: a randomized, double-blind, placebo-controlled trial. Addiction, 2013. 108(4): p. 751-61.
30. Strazza, M., et al., Breaking down the barrier: the effects of HIV-1 on the blood-brain barrier. Brain Res, 2011. 1399: p. 96-115.
31. Yao, H., et al., HV neuropathogenesis: a tight rope walk of innate immunity. J Neuroimmune Pharmacol, 2010. 5(4): p. 489-95.
32. Kraft-Terry, S.D., et al., MV-1 neuroimmunity in the era of antiretroviral therapy.

Neurobiol Dis, 2010. 37(3): p. 542-8.
33. Arseniou, S., A. Arvaniti, and M. Samakouri, HV infection and depression.

Psychiatry Clin Neurosci, 2014. 68(2): p. 96-109.
34. Del Guerra, F. B., et al., Human immunodeficiency virus-associated depression: contributions of immuno-inflammatory, monoaminergic, neurodegenerative, and neurotrophic pathways. J Neurovirol, 2013. 19(4): p. 314-27.
35. Benton, T. D., Depression and HIV/AIDS. Curr Psychiatry Rep, 2008. 10(3): p. 280-5.
36. Goncalves, J., et al., Methamphetamine-induced neuroinflammation and neuronal dysfunction in the mice hippocampus: preventive effect of indomethacin. Eur J Neurosci. 31(2): p. 315-26.
37. LaVoie, M. J., J. P. Card, and T. G. Hastings, Microglial activation precedes dopamine terminal pathology in methamphetamine-induced neurotoxicity. Exp Neurol, 2004. 187(1): p. 47-57.
38. Thomas, D. M., D. M. Francescutti-Verbeem, and D. M. Kuhn, Methamphetamine-induced neurotoxicity and microglial activation are not mediated by fractalkine receptor signaling. J Neurochem, 2008. 106(2): p. 696-705.
39. Sekine, Y., et al., Methamphetamine causes microglial activation in the brains of human abusers. J Neurosci, 2008. 28(22): p. 5756-61.
40. Thomas, D. M., D. M. Francescutti-Verbeem, and D. M. Kuhn, The newly synthesized pool of dopamine determines the severity of methamphetamine-induced neurotoxicity. J Neurochem, 2008. 105(3): p. 605-16.
41. Kitamura, O., Detection of methamphetamine neurotoxicity in forensic autopsy cases. Leg Med (Tokyo), 2009. 11 Suppl 1: p. S63-5.
42. Buchanan, J. B., N. L. Sparkman, and R. W. Johnson, A neurotoxic regimen of methamphetamine exacerbates the febrile and neuroinflammatory response to a subsequent peripheral immune stimulus. J Neuroinflammation. 7: p. 82.
43. Farahzadi, M. H., et al., Riluzole for treatment of men with methamphetamine dependence: A randomized, double-blind, placebo-controlled clinical trial. J Psychopharmacol, 2019. 33(3): p. 305-315.
44. Brown, E. S. and B. Gabrielson, A randomized, double-blind, placebo-controlled trial of citicoline for bipolar and unipolar depression and methamphetamine dependence.

J Affect Disord, 2012. 143(1-3): p. 257-60.
45. Johnson, B. A., et al., A preliminary randomized, double-blind, placebo-controlled study of the safety and efficacy of ondansetron in the treatment of methamphetamine dependence. Int J Neuropsychopharmacol, 2008. 11(1): p. 1-14.
46. Ling, W., et al., Double-blind placebo-controlled evaluation of the PROMETA protocol for methamphetamine dependence. Addiction, 2012. 107(2): p. 361-9.
47. Peterson, E. C., W. B. Gentry, and S. M. Owens, Customizing monoclonal antibodies for the treatment of methamphetamine abuse: current and future applications. Adv Pharmacol, 2014. 69: p. 107-27.
48. Ferris, M. J., C. F. Mactutus, and R. M. Booze, Neurotoxic profiles of HIV, psychostimulant drugs of abuse, and their concerted effect on the brain: current status of dopamine system vulnerability in NeuroAIDS. Neurosci Biobehav Rev, 2008. 32(5): p. 883-909.
49. Moran, L. M., et al., Adolescent HIV-1 transgenic rats: evidence for dopaminergic alterations in behavior and neurochemistry revealed by methamphetamine challenge.

Curr HIV Res, 2012. 10(5): p. 415-24.
50. Liu, Z., et al., HIV transactivator of transcription enhances methamphetamine-induced Parkinson's-like behavior in the rats. Neuroreport, 2014.
51. Kuhn, D. M., D. M. Francescutti-Verbeem, and D. M. Thomas, Dopamine quinones activate microglia and induce a neurotoxic gene expression profile: relationship to methamphetamine-induced nerve ending damage. Ann N Y Acad Sci, 2006. 1074: p. 31-41.
52. Maragos, W. F., et al., Human immunodeficiency virus-1 Tat protein and methamphetamine interact synergistically to impair striatal dopaminergic function. J Neurochem, 2002. 83(4): p. 955-63.
53. Cass, W. A., et al., HIV-1 protein Tat potentiation of methamphetamine-induced decreases in evoked overflow of dopamine in the striatum of the rat. Brain Res, 2003. 984(1-2): p. 133-42.
54. Tiihonen, J., et al., A comparison of aripiprazole, methylphenidate, and placebo for amphetamine dependence. Am J Psychiatry, 2007. 164(1): p. 160-2.
55. Fantegrossi, W. E., et al., A comparison of the physiological, behavioral, neurochemical and microglial effects of methamphetamine and 3,4-methylenedioxymethamphetamine in the mouse. Neuroscience, 2008. 151(2): p. 533-43.
56. O'Callaghan, J. P., K. Sriram, and D. B. Miller, Defining “neuroinflammation”.

Ann N Y Acad Sci, 2008. 1139: p. 318-30.
57. Cadet, J. L., et al., Neurotoxicity of substituted amphetamines: molecular and cellular mechanisms. Neurotox Res, 2007. 11(3-4): p. 183-202.
58. Ren, Q., et al., 7,8-Dihydroxyflavone, a TrkB agonist, attenuates behavioral abnormalities and neurotoxicity in mice after administration of methamphetamine.

Psychopharmacology (Berl), 2014. 231(1): p. 159-66.
59. Ren, Q., et al., Effects of TrkB agonist 7,8-dihydroxyflavone on sensory gating deficits in mice after administration of methamphetamine. Pharmacol Biochem Behav, 2013. 106: p. 124-7.
60. Jan, R. K., et al., Striatal volume increases in active methamphetamine-dependent individuals and correlation with cognitive performance. Brain Sci, 2012. 2(4): p. 553-72.
61. Runarsdottir, V., et al., Extended-Release Injectable Naltrexone (XR-NTX) With Intensive Psychosocial Therapy for Amphetamine-Dependent Persons Seeking Treatment: A Placebo-Controlled Trial. J Addict Med, 2017. 11(3): p. 197-204.
62. Coffin, P. O., et al., Extended-release naltrexone for methamphetamine dependence among men who have sex with men: a randomized placebo-controlled trial. Addiction, 2018. 113(2): p. 268-278.
63. Thomas, D. M., D. M. Francescutti-Verbeem, and D. M. Kuhn, Increases in cytoplasmic dopamine compromise the normal resistance of the nucleus accumbens to methamphetamine neurotoxicity. J Neurochem, 2009. 109(6): p. 1745-55.
64. Kuhn, D. M., D. M. Francescutti-Verbeem, and D. M. Thomas, Dopamine disposition in the presynaptic process regulates the severity of methamphetamine-induced neurotoxicity. Ann N Y Acad Sci, 2008. 1139: p. 118-26.
65. Aron, J. L. and M. P. Paulus, Location, location: using functional magnetic resonance imaging to pinpoint brain differences relevant to stimulant use. Addiction, 2007. 102 Suppl 1: p. 33-43.
66. Morein-Zamir, S. and T. W. Robbins, Fronto-striatal circuits in response-inhibition: Relevance to addiction. Brain Res, 2015. 1628(Pt A): p. 117-29.
67. Rothman, R. B., et al., Neurochemical neutralization of methamphetamine with high-affinity nonselective inhibitors of biogenic amine transporters: a pharmacological strategy for treating stimulant abuse. Synapse, 2000. 35(3): p. 222-7.
68. Preti, A., Vanoxerine National Institute on Drug Abuse. Curr Opin Investig Drugs, 2000. 1(2): p. 241-51.
69. Sogaard, U., et al., A tolerance study of single and multiple dosing of the selective dopamine uptake inhibitor GBR 12909 in healthy subjects. Int Clin Psychopharmacol, 1990. 5(4): p. 237-51.
70. Glowa J R, S.D., Rice K C, and Rothman R B, Effects of GBR12909 on responding maintained under a progressive ratio schedule of methamphetamine delivery in rhesus monkeys. Drug Alcohol Depend, 2001. 63: p. S55-S56.
71. Dittrich, H. C., et al., COR-ART: A multicenter, randomized, double-blind, placebo-controlled dose-ranging study to evaluate single oral doses of vanoxerine for conversion of recent-onset atrial fibrillation or flutter to normal sinus rhythm. Heart Rhythm, 2015. 12(6): p. 1105-12.
72. Illum, L., Nasal drug delivery—recent developments and future prospects. J Control Release, 2012. 161(2): p. 254-63.
73. Mehrafza, S., et al., Pharmacological evidence for lithium-induced neuroprotection against methamphetamine-induced neurodegeneration via Akt-1/GSK3 and CREB-BDNF signaling pathways. Iran J Basic Med Sci, 2019. 22(8): p. 856-865.
74. Keshavarzi, S., et al., Protective role of metformin against methamphetamine induced anxiety, depression, cognition impairment and neurodegeneration in rat: The role of CREB/BDNF and Akt/GSK3 signaling pathways. Neurotoxicology, 2019. 72: p. 74-84.
75. Johnson-Farley, N. N., et al., Interaction of FGF-2 with IGF-1 and BDNF in stimulating Akt, ERK, and neuronal survival in hippocampal cultures. Brain Res, 2007. 1154: p. 40-9.
76. Zheng, W. H. and R. Quirion, Comparative signaling pathways of insulin-like growth factor-1 and brain-derived neurotrophic factor in hippocampal neurons and the role of the PI3 kinase pathway in cell survival. J Neurochem, 2004. 89(4): p. 844-52.
77. Johnson-Farley, N. N., T. Travkina, and D. S. Cowen, Cumulative activation of akt and consequent inhibition of glycogen synthase kinase-3 by brain-derived neurotrophic factor and insulin-like growth factor-1 in cultured hippocampal neurons. J Pharmacol Exp Ther, 2006. 316(3): p. 1062-9.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed:

1. A composition comprising:
(i) a dopamine active transporter (DAT) targeting agent comprising a dopamine re-uptake inhibitor (DRI) or an anti-DAT antibody; (ii) a neurotrophin; and (iii) a carrier that protects the composition against rapid elimination from the body of the living subject,
wherein the DAT targeting agent and neurotrophin are linked to a clathrin nanoparticle.

2. The composition of claim 1, wherein the DRI is selected from the group consisting of -Hydroxy-1-methyl-4-(4-methylphenyl)-3-piperidyl 4-methylphenyl ketone, Altropane (O-587), Amfonelic acid (WIN 25978), Amineptine, BTCP (GK-13), 3C-PEP, DBL-583, Difluoropine (O-620), GBR-12783, GBR-12935, GBR-13069, GBR-13098, GYKI-52895, Iometopane (β-CIT, RTI-55), Methylphenidate, Ethylphenidate, Modafinil, Armodafinil, RTI-229, Vanoxerine (GBR-12909), drafinil, Amantadine, Benztropine, Bupropion, Fluorenol, Ketamine, Medifoxamine, Metaphit, Rimcazole, Sertraline, St. John's Wort, Venlafaxine, Chaenomeles speciose, 3-Methoxyphencyclidine, 4-Methoxyphencyclidine, and Oroxylin A.

3. The composition of claim 1, wherein the neurotrophin comprises brain-derived neurotrophic factor (BDNF), nerve growth factor (NGF), neurotrophin-3 (NT-3), neurotrophin-4 (NT-4), neurotrophin-6 (NT-6), glial cell line-derived neurotrophic factor (GDNF), neurturin (NTN), persephin (PSPN), artemin (ARTN), ciliary neurotrophic factor (CNTF), or leukemia inhibitory factor (LIF).

4. The composition of claim 1, wherein the carrier is a biodegradable, biocompatible polymer.

5. The composition of claim 4, wherein the carrier comprises ethylene vinyl acetate, polyanhydrides, polyglycolic acid, collagen, polyorthoesters, or polylactic acid

6. The composition of claim 1, wherein the composition is prepared as a controlled release formulation.

7. The composition of claim 6, wherein the composition comprises an implant or a microencapsulated delivery system.

8. The composition of claim 1, wherein the DAT targeting agent and/or the neurotrophin is linked to the clathrin nanoparticle by conjugation.

9. The composition of claim 8, wherein the DAT targeting agent is conjugated to the clathrin nanoparticle via polyethylene glycol (PEG).

10. The composition of claim 8, wherein the neurotrophin is conjugated to the clathrin nanoparticle via PEG.

11. A method for treating a human subject having or at risk for developing a dopamine disorder comprising administering to the human subject a therapeutically effective amount of the composition of claim 1.

12. The method of claim 11, wherein the dopamine disorder is a neurodegenerative disease associated with loss of dopaminergic neurons.

13. The method of claim 12, wherein the neurodegenerative disease associated with loss of dopaminergic neurons is Parkinson's disease, Huntington's disease, Alzheimer's disease, or ALS.

14. The method of claim 11, wherein the dopamine disorder is ADHD, depression, or schizophrenia.

15. The method of claim 11, wherein the dopamine disorder is a dopamine-driven addiction.

16. The method of claim 11, wherein the composition is delivered intranasally or intravenously.
